(19) United States
(12) Patent Application Publication
(10) Pub. No.: US 2026/0334756 A1
(43) Pub. Date: Oct. 29, 2026

(54) SYSTEMS AND METHODS FOR ENDOSCOPIC VIDEO VISUALIZATION USING A HEAD-MOUNTED DISPLAY

(71) Applicant: THE FEINSTEIN INSTITUTES FOR MEDICAL RESEARCH, INC., Manhasset, NY (US)

(72) Inventors: Phillip CONNELL, Manhasset, NY (US); Johnny ZHANG, Manhasset, NY (US); Michael RECINE, Manhasset, NY (US); Timir B. DATTA-CHAUDHURI, Manhasset, NY (US)

(73) Assignee: THE FEINSTEIN INSTITUTES FOR MEDICAL RESEARCH, INC., Manhasset, NY (US)

(21) Appl. No.: 19/651,887

(22) Filed: Apr. 20, 2026

Related U.S. Application Data
(60) Provisional application No. 63/793,047, filed on Apr. 23, 2025.

(51) Int. Cl. A61B 1/00; A61B 1/04; A61B 1/06; A61B 1/313

(52) U.S. Cl. CPC A61B 1/00048; A61B 1/04; A61B 1/0661; A61B 1/3132

(57) ABSTRACT

An endoscopic video visualization system includes a video capture source releasably couplable to an endoscope, a receiver module separate from the video capture source, and a user interface communicatively coupled to the receiver module. At least one processor of the receiver module may receive endoscopic video data from the video capture source over a network connection in accordance with a streaming protocol, may process the endoscopic video data by decoding the endoscopic video data when the endoscopic video data is received in a compressed format, may generate output data for presentation on the user interface based on the endoscopic video data, and may transmit the output data over a wired or wireless connection to the user interface. The head-mounted display may present the output data within a line of sight of a user during a scope-based procedure.

### FIELD OF TECHNOLOGY

The present disclosure relates to systems and methods for endoscopic video visualization and, more particularly, to systems and methods for endoscopic video visualization using a head-mounted display in laparoscopy.

### BACKGROUND OF TECHNOLOGY

Laparoscopic and other endoscopic procedures commonly rely on visualization systems that capture video from an endoscope and present the video to a clinician during a procedure. As minimally invasive techniques continue to expand across surgical settings, there has been an increasing need for visualization platforms that support reliable image presentation in a manner compatible with the clinical workflow of an operating room (OR). These environments may involve constrained physical space, sterile field considerations, multiple pieces of equipment, and demands for continuous visualization while clinicians perform precise procedural tasks.

Conventional surgical visualization arrangements may rely on equipment that occupies substantial space in the operating room, includes multiple physical connections, and presents video on displays positioned away from the procedural site. As a result, clinicians may experience ergonomic constraints, workflow inefficiencies, setup burdens, and practical limitations associated with portability, infrastructure requirements, and use in resource-constrained environments. In addition, maintaining high-quality video presentation with low delay and consistent reliability during endoscopic procedures remains an ongoing technical challenge.

Thus, there is a need in the art for improved endoscopic visualization technologies that support efficient clinical workflow, reduced equipment burden, and dependable video presentation during minimally invasive procedures.

### SUMMARY

In at least some embodiments, a method may include receiving, by at least one processor of a receiver module that may be separate from a video capture source releasably coupled to an endoscope, endoscopic video data transmitted from the video capture source over a network connection in accordance with a streaming protocol, processing, by the at least one processor, the endoscopic video data by decoding the endoscopic video data when the endoscopic video data may be received in a compressed format, generating, by the at least one processor and based on the endoscopic video data, output data for presentation on a head-mounted display, transmitting, by the at least one processor, the output data over a wired connection to the head-mounted display, and causing, by the at least one processor, presentation of the output data by the head-mounted display within a line of sight of a user during a scope-based procedure.

In at least some embodiments, a receiver module may include a non-transitory memory storing computer code and at least one processor in communication with the non-transitory memory, where the at least one processor may be configured to execute the computer code to cause the receiver module to receive endoscopic video data from a video capture source releasably couplable to an endoscope over a network connection in accordance with a streaming protocol, to process the endoscopic video data by decoding the endoscopic video data when the endoscopic video data may be received in a compressed format, to generate output data for presentation on a head-mounted display based on the endoscopic video data, and to transmit the output data over a wired connection to the head-mounted display for presentation within a line of sight of a user during a scope-based procedure, where the receiver module may be separate from the video capture source.

In at least some embodiments, a system may include a video capture source releasably couplable to an endoscope, a receiver module separate from the video capture source, and a head-mounted display communicatively coupled to the receiver module, where the receiver module may include a non-transitory memory storing computer code and at least one processor in communication with the non-transitory memory, and where the at least one processor may be configured to execute the computer code to cause the receiver module to receive endoscopic video data from the video capture source over a network connection in accordance with a streaming protocol, to process the endoscopic video data by decoding the endoscopic video data when the endoscopic video data may be received in a compressed format, to generate output data for presentation on the head-mounted display based on the endoscopic video data, and to transmit the output data over a wired connection to the head-mounted display, where the head-mounted display may be configured to present the output data within a line of sight of a user during a scope-based procedure.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 is a system depicting an endoscope-based visualization arrangement in accordance with one or more embodiments of the present disclosure;

FIG. 2 is a block diagram depicting an endoscopic visualization system in accordance with one or more embodiments of the present disclosure;

FIG. 3 is a schematic depicting a smartphone as a receiver module in accordance with one or more embodiments of the present disclosure;

FIG. 4 is a schematic depicting a Pocket IXR device as a receiver module in accordance with one or more embodiments of the present disclosure;

FIG. 5 is a schematic depicting an IXR smartphone dock/server implementation with a separate receiver for XR glasses in accordance with one or more embodiments of the present disclosure;

FIG. 6 is a schematic depicting an IXR box/server receiver architecture in accordance with one or more embodiments of the present disclosure;

FIG. 7 is a schematic depicting an IXR camera with receiver system in accordance with one or more embodiments of the present disclosure;

FIG. 8 is a schematic depicting a legacy laparoscopic tower implementation in which endoscopic video data may be provided by a tower camera and may be transmitted by a transmission module to a smartphone receiver module for output to XR glasses in accordance with one or more embodiments of the present disclosure;

FIG. 9 is a schematic depicting an IXR transmission module visualization architecture with a Pocket IXR receiver in accordance with one or more embodiments of the present disclosure;

FIG. 10 is a schematic depicting an IXR box setup with a smartphone in accordance with one or more embodiments of the present disclosure;

FIG. 11 is a schematic depicting an IXR box setup with an IXR pocket receiver in accordance with one or more embodiments of the present disclosure;

FIG. 12 is an exploded view depicting a wireless camera in accordance with one or more embodiments of the present disclosure;

FIG. 13 is an exploded view depicting a light source assembly for an endoscope in accordance with one or more embodiments of the present disclosure;

FIG. 14 is an exploded view depicting a receiver module in accordance with one or more embodiments of the present disclosure;

FIG. 15A is a view of a housing architecture in accordance with one or more embodiments of the present disclosure;

FIG. 15B depicts at least a portion of a sterile housing/accessory in an exploded arrangement in accordance with one or more embodiments of the present disclosure;

FIG. 16 is an exploded view depicting a non-sterile camera in accordance with one or more embodiments of the present disclosure;

FIG. 17 is an exploded view depicting a sterile housing assembly in accordance with one or more embodiments of the present disclosure; and

FIG. 18 is a process flow depicting an aseptic handling technique in accordance with one or more embodiments of the present disclosure.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

Endoscopic procedures such as laparoscopy may rely on capture and display of live video from an endoscope to enable a clinician to perform a procedure safely and efficiently. In many clinical settings, existing visualization arrangements may impose technical constraints that may include limited portability, burdensome setup, extensive cabling, and a display location that may be outside a natural line of sight of the clinician, which may contribute to ergonomic strain and workflow inefficiency.

In addition, providing reliable, low-latency delivery of endoscopic video across an operating room environment may present technical challenges, particularly where equipment may need to be positioned inside or outside a sterile field, where network conditions may vary, and where a clinician may benefit from hands-free or minimally intrusive user interaction while maintaining continuous visualization.

At least some embodiments of the present disclosure herein disclose systems and methods that may capture endoscopic video using a video capture source associated with an endoscope, may transmit endoscopic video data using a low-latency streaming protocol over a network connection, may receive and may process the endoscopic video data at a receiver module, and may output video or other output data over a wired connection for presentation on a head-mounted display within a line of sight of a user during a scope-based procedure.

In at least some embodiments, a modular architecture may include a light source, an endoscope, a video capture source, a streaming protocol, a network connection, a receiver module, and a user interface or user experience (UI/UX) output, where each module may be selected, substituted, or configured independently to support different procedural environments and different equipment constraints.

In at least some embodiments, the light source may include an illumination module that may physically attach to a light port of an endoscope and may illuminate a body cavity, where the light source may include one or more light emitting diodes and may be battery-powered to reduce cabling and operating room clutter.

In at least some embodiments, the light source may include one or more light emitters that may be operated in a strobed mode during image capture, where the one or more light emitters may be pulsed in timed relation with exposure of an image sensor of the video capture source so that illumination may be provided during a selected exposure window rather than continuously throughout operation. In at least some embodiments, a control circuit of the light source, the video capture source, or another cooperating device may control pulse timing, pulse duration, pulse frequency, and pulse intensity based on a frame rate, an exposure setting, an imaging mode, and/or a procedure condition.

In at least some embodiments, synchronization between the light source and the video capture source may be achieved using a timing signal, a trigger signal, a clock signal, software control, hardware control, or any combination thereof, where the timing signal may indicate initiation of a frame capture event or another image acquisition interval. In at least some embodiments, the one or more light emitters may be activated before, during, and/or for at least a portion of an exposure interval of the image sensor and may be deactivated outside the exposure interval so that emitted light may be concentrated during acquisition of endoscopic video data.

In at least some embodiments, operation of the light source in the strobed mode may reduce average power consumption of the light source relative to continuous illumination, where a battery associated with the light source and/or the video capture source may thereby support an extended duration of use during a scope-based procedure. In at least some embodiments, the strobed mode may additionally reduce average heat generation at the light source and near the endoscope while still permitting a selected level of perceived scene brightness and image quality.

In at least some embodiments, because the one or more light emitters may be driven for less than a full frame period, the one or more light emitters may be operated at a higher instantaneous output level during the exposure interval than may be practical during continuous operation, where the higher instantaneous output level may support image capture with increased illumination while maintaining thermal and power conditions within a desired operating range. In at least some embodiments, synchronization of strobed illumination with frame capture may support improved visualization of anatomical structures and/or procedural instruments while preserving portability of a battery-powered illumination platform.

In at least some embodiments, the video capture source may include a camera module that may physically attach to an eyepiece portion of an endoscope and may acquire endoscopic video at a resolution of about 1080p or higher, where the video capture source may include a data transmission module that may transmit endoscopic video data to a receiver module over a wired connection or a wireless connection.

In at least some embodiments, the video capture source may include a hardware-based encoder configured to encode endoscopic video data before transmission to the receiver module, where the hardware-based encoder may be included in the video capture source and may operate as part of a camera module, a single board computer, an image-processing pipeline, a system on a chip, an application-specific integrated circuit, a field programmable gate array, and/or another dedicated hardware arrangement associated with the video capture source.

In at least some embodiments, the hardware-based encoder may include an intraframe encoder configured to encode individual image frames independently of one another, where the intraframe encoder may include a Motion JPEG (MJPEG) encoder and/or another intraframe compression arrangement selected to support low-latency handling of live endoscopic video data. In at least some embodiments, use of intraframe encoding at the video capture source may reduce interframe dependency and may support responsive decoding, frame access, and downstream presentation at the receiver module and the head-mounted display.

In at least some embodiments, the video capture source may generate compressed endoscopic video data using the hardware-based encoder and may transmit the compressed endoscopic video data over the network connection in accordance with the streaming protocol, where the receiver module may receive the compressed endoscopic video data and may decode the compressed endoscopic video data for generation of output data for presentation on the head-mounted display. In at least some embodiments, the compressed endoscopic video data may include data encoded using MJPEG, H.264, and/or another compression format selected based on latency, bandwidth, image quality, compatibility, compute availability, and/or workflow requirements.

In at least some embodiments, implementation of the hardware-based intraframe encoder at the video capture source may reduce processing burden and/or latency associated with live endoscopic video streaming relative to software-based encoding performed at a general-purpose processing device, where the hardware-based intraframe encoder may support portable operation, real-time or near-real-time transmission, and reliable presentation of endoscopic video within a line of sight of a user during a scope-based procedure.

In at least some embodiments, the streaming protocol may determine how endoscopic video data may be transmitted between the video capture source and the receiver module, where the streaming protocol may be selected to support low-latency transmission for compressed data or uncompressed data, and where the streaming protocol may include a low latency protocol, a Real-Time Streaming Protocol (RTSP), and/or a User Datagram Protocol (UDP).

In at least some embodiments, the network connection may include infrastructure over which endoscopic video data may be transmitted, where the network connection may include a wired network and/or a wireless network, and where the network connection may be configured to be stable, secure, and to provide bandwidth appropriate for the selected streaming protocol.

In at least some embodiments, the receiver module may include a device that may receive endoscopic video data via the streaming protocol over the network connection and may output video or output data to a UI/UX module, where the receiver module may be positioned on a user or off a user, and where the receiver module may include a smartphone, a tablet, an Immersive Extended Reality (IXR) device, an IXR smartphone dock, a data hub, a server, and/or other receiver-side hardware.

In at least some embodiments, the UI/UX module may include a head-mounted display that may include extended reality (XR) glasses that may present endoscopic video within the line of sight of the user, where the UI/UX module additionally or alternatively may include a mobile application, a monitor, and/or a browser, and where the UI/UX module may include an audio interface for user interaction.

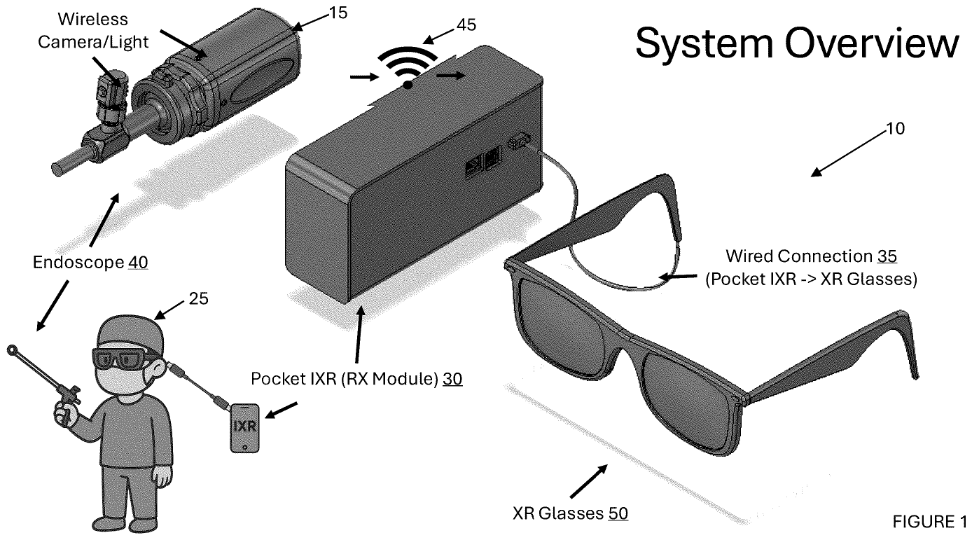

FIG. 1 is a system 10 depicting an endoscope-based visualization arrangement in accordance with one or more embodiments of the present disclosure, where the arrangement may include a wireless camera/light 15, a user 25, such as for example, a medical practitioner, an endoscope 40, a Pocket IXR receiver module 30, a wireless connection 45 between the wireless camera/light 15 and the Pocket IXR receiver module 30, a wired connection 35 between the Pocket IXR receiver module 30 and XR glasses 50, and the XR glasses 50.

In at least some embodiments, the wireless camera/light 15 may include a light source portion and a video capture source portion integrated into a common assembly, where the light source portion may physically attach to a light port of the endoscope 40 and may illuminate a body cavity being operated in, and where the video capture source portion may physically attach to an eyepiece portion of the endoscope 40 and may capture endoscopic video and/or still images.

In at least some embodiments, the endoscope 40 may include any suitable endoscope used in a scope-based procedure, and the wireless camera/light 15 may be removably coupled to the endoscope 40 such that the endoscope 40 may provide an optical pathway to the body cavity while the wireless camera/light 15 may provide illumination and image capture functionality.

In at least some embodiments, the wireless camera/light 15 may include a data transmission module that may transmit endoscopic video data over the wireless network connection 45 using a low-latency streaming protocol, where the streaming protocol may include compressed or uncompressed transmission and may include, for example, a low latency protocol, RTSP, and/or UDP.

In at least some embodiments, the Pocket IXR receiver module 30 may receive the endoscopic video data transmitted from the wireless camera/light 15, and the Pocket IXR receiver module 30 may include dedicated receiver-side hardware that may be optimized for onboard encoding, decoding, edge computing, and/or artificial intelligence processing. In at least some embodiments, the Pocket IXR receiver module 30 may be portable, may be wearable on a user, and may be positioned on the surgeon rather than off the sterile field.

In at least some embodiments, the wired connection 35 may extend between the Pocket IXR receiver module 30 and the XR glasses 50 and may carry video and/or output data for presentation to the user, where the wired connection 35 may include a USB-C connection to support low-latency delivery of the output data to the XR glasses 50.

In at least some embodiments, the XR glasses 50 may include a head-mounted display that may present endoscopic video within a line of sight of the user during a scope-based procedure, where the XR glasses 50 may be off-the-shelf and vendor agnostic, and where the XR glasses 50 may include an audio interface and/or an external camera in some implementations.

In at least some embodiments, FIG. 1 may illustrate a signal path in which the wireless camera/light 15 may capture and wirelessly transmit endoscopic video data from the endoscope 40 to the Pocket IXR receiver module 30, and the Pocket IXR receiver module 30 may output corresponding video or output data over the wired connection 35 to the XR glasses 50 for display to the user.

In at least some embodiments, the arrangement shown in FIG. 1 may correspond to a Pocket IXR receiver implementation of a broader modular platform architecture in which the wireless camera/light 15 may function as at least part of a light source and a video capture source, the wireless transmission path may function as a streaming protocol and network connection, the Pocket IXR receiver module 30 may function as a receiver module, and the XR glasses 50 may function as a UI/UX output.

In at least some embodiments, the arrangement shown in FIG. 1 may provide a portable visualization platform that may reduce reliance on legacy laparoscopic tower displays, may reduce operating room clutter, and may present endoscopic video within the user's natural line of sight while maintaining a separation between capture hardware at the endoscope 40 and display hardware at the XR glasses 50.

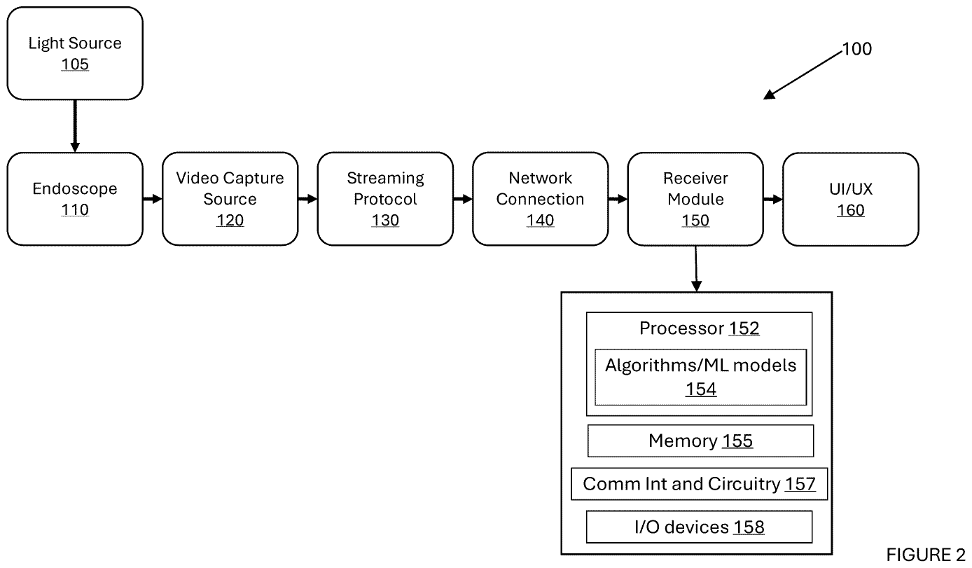

FIG. 2 is a block diagram depicting an endoscopic visualization system 100 in accordance with one or more embodiments of the present disclosure, where the system 100 may include a light source 105, an endoscope 110, a video capture source 120, a streaming protocol 130, a network connection 140, a receiver module 150, and a user interface/user experience (UI/UX) output 160. In at least some embodiments, the receiver module 150 may further include a processor 152, algorithms and/or machine learning models 154, memory 155, communication interface and circuitry 157, and input/output devices 158.

In at least some embodiments, the light source 105 may include an illumination module configured to provide light to the endoscope 110 for illuminating a body cavity or other anatomical region during a scope-based procedure. The light source 105 may include one or more light emitting diodes, may be battery-powered, and may physically attach to a light port of the endoscope 110. In some implementations, the light source 105 may be integrated with the video capture source 120 in a common housing.

In at least some embodiments, the endoscope 110 may include a rigid or flexible endoscope configured to provide an optical pathway to a surgical or diagnostic site. The endoscope 110 may be used in laparoscopy and may also be used in other scope-based procedures. The endoscope 110 may interface mechanically and optically with the video capture source 120 and may interface mechanically and optically with the light source 105.

In at least some embodiments, the video capture source 120 may include a camera module configured to attach to an eyepiece portion of the endoscope 110 and to acquire endoscopic video and/or still images. The video capture source 120 may include image sensing hardware, onboard compute resources, power resources, and transmission circuitry. The video capture source 120 may transmit endoscopic video data over a wired or wireless path toward the receiver module 150.

In at least some embodiments, the streaming protocol 130 may define how endoscopic video data may be formatted and transmitted between the video capture source 120 and the receiver module 150. The streaming protocol 130 may be selected to support low-latency delivery of compressed data or uncompressed data. The streaming protocol 130 may include, for example, a Real-Time Streaming Protocol (RTSP), a User Datagram Protocol (UDP), and/or another low-latency streaming protocol.

In at least some embodiments, the network connection 140 may include communication infrastructure over which the endoscopic video data may travel from the video capture source 120 to the receiver module 150. The network connection 140 may include a wireless network, a wired network, a local area network, an access point connection, and/or another communication path that may support bandwidth and latency characteristics appropriate for live endoscopic video.

In at least some embodiments, the receiver module 150 may include a receiver-side device configured to receive the endoscopic video data via the streaming protocol 130 over the network connection 140 and to output video or output data to a user interface. The receiver module 150 may be positioned on a user or off a user and may include, for example, a smartphone, a Pocket IXR device, an IXR smartphone dock, an IXR box, a data hub, a tablet, a server, and/or another receiver-side hardware implementation.

In at least some embodiments, the processor 152 may include at least one processor configured to execute computer code for handling the endoscopic video data received by the receiver module 150. The processor 152 may receive the endoscopic video data, may process the endoscopic video data, may decode the endoscopic video data when the endoscopic video data may be received in a compressed format, may generate output data for presentation on a head-mounted display, and may transmit the output data to the head-mounted display and/or another user interface.

In at least some embodiments, the algorithms and/or machine learning models 154 may include software routines, computer vision routines, artificial intelligence routines, predictive routines, and/or other processing logic that may be executed by the processor 152. The algorithms and/or machine learning models 154 may support image handling, object detection, voice-activated support, natural language processing, workflow support, and/or other advanced processing functions associated with the receiver module 150.

In at least some embodiments, the memory 155 may include a non-transitory memory configured to store computer code, configuration data, streaming information, recorded video data, processed output data, algorithm parameters, and/or machine learning model data. The memory 155 may be communicatively coupled to the processor 152 and may support execution of receiver-side operations.

In at least some embodiments, the communication interface and circuitry 157 may include wired and/or wireless interface hardware configured to support communication between the receiver module 150 and external devices. The communication interface and circuitry 157 may support reception of the endoscopic video data from the video capture source 120 over the network connection 140 and may support output of video or output data toward XR glasses, a monitor, a browser-based interface, a mobile application, and/or another output device.

In at least some embodiments, the input/output devices 158 may include ports, connectors, controls, and interface devices associated with the receiver module 150. The input/output devices 158 may include a wired output port such as a USB-C port for connection to XR glasses, and may further include user input devices, display devices, audio devices, and/or interface hardware that may support interaction with the system 100.

In at least some embodiments, FIG. 2 may illustrate a modular architecture in which the light source 105, endoscope 110, video capture source 120, streaming protocol 130, network connection 140, and receiver module 150 may be selected and configured independently while remaining interoperable within the system 100. Such modularity may permit different hardware implementations and workflow configurations while preserving a common functional pipeline for capturing, transmitting, receiving, processing, and presenting endoscopic video.

In at least some embodiments, FIG. 2 may further illustrate that processing responsibility may be centered at the receiver module 150 through the processor 152, the algorithms and/or machine learning models 154, the memory 155, the communication interface and circuitry 157, and the input/output devices 158, while image acquisition may be centered at the video capture source 120 and optical access may be centered at the endoscope 110 with illumination support from the light source 105.

In at least some embodiments, the receiver module 150 may include a portable or non-portable computing device configured to receive endoscopic video data from the video capture source, to process the endoscopic video data, and to output video or other output data to a user interface. The receiver module 150 may include a smartphone, a tablet, a Pocket IXR device, an IXR smartphone dock, an IXR box, a data hub, a server, XR glasses, or another receiver-side hardware platform, and may be positioned on a user, worn by a user, carried by a user, mounted in an operating room, positioned off a sterile field, or distributed across two or more cooperating devices.

In at least some embodiments, the processor 152 may include one or more processing resources configured to execute computer code and to perform receiver-side operations. The processor 152 may include a central processing unit, a graphics processing unit, a neural processing unit, a digital signal processor, a field programmable gate array, an application specific integrated circuit, a microprocessor, a processor having multiple processing units, a system on a chip, or any combination thereof. The processor 152 may perform one or more operations that may include receiving streamed endoscopic video data, decoding compressed video data, encoding video data, generating output data for presentation on a head-mounted display, performing edge computing, performing artificial intelligence processing, managing communications, controlling peripherals, and coordinating real-time or near-real-time workflow functions.

In at least some embodiments, the algorithms and/or machine learning models 154 may include software, firmware, logic, heuristics, rules, trained models, and/or model parameters stored in memory and executable by the processor 152. The algorithms and/or machine learning models 154 may include video-processing algorithms, image enhancement algorithms, compression and decompression algorithms, computer vision algorithms, object detection algorithms, tracking algorithms, natural language processing algorithms, speech or voice recognition algorithms, workflow-support algorithms, predictive models, recommendation logic, and/or agentic artificial intelligence routines. Such algorithms and/or machine learning models 154 may support endoscopic video visualization, object detection, contextual assistance, voice-activated control, natural language interaction, remote consultation support, surgical workflow support, clinical documentation support, and/or postoperative note generation.

In at least some embodiments, the algorithms and/or machine learning models 154 may be executed locally at the receiver module 150, at least partly at the video capture source, at an IXR smartphone dock, at an IXR box, at a server, in a cloud-computing environment, or across any combination of cooperating devices. The algorithms and/or machine learning models 154 may be updated, replaced, retrained, fine-tuned, or selectively invoked based on available compute resources, latency constraints, network conditions, procedure type, user preferences, and/or clinical workflow requirements. In some implementations, the algorithms and/or machine learning models 154 may be trained using stored surgical videos, including laparoscopic procedure videos, and may thereafter be used for inference during live procedures.

In at least some embodiments, the memory 155 may include one or more non-transitory computer-readable storage media configured to store executable instructions, configuration data, user settings, video buffers, temporary data, recorded video, output data, metadata, event logs, procedure data, model weights, and/or other information used by the receiver module 150. The memory 155 may include volatile memory, non-volatile memory, random access memory, flash memory, solid-state memory, read only memory, removable memory, local storage, network-attached storage, cloud storage, or any combination thereof.

In at least some embodiments, the communication interface and circuitry 157 may include wired and/or wireless hardware and software configured to support data exchange between the receiver module 150 and one or more external devices. The communication interface and circuitry 157 may support communication using a local area network, a wide area network, the Internet, a virtual private network, a wireless access point, Ethernet, USB, Bluetooth, Wi-Fi, cellular communication, near-field communication, and/or other wired or wireless communication modes. The communication interface and circuitry 157 may support reception of endoscopic video data from the video capture source in accordance with a streaming protocol that may include a low latency streaming protocol, RTSP, UDP, or another protocol, and may support output of video or output data to XR glasses, a monitor, a browser-based interface, a mobile application, a remote consultation endpoint, and/or another local or remote device.

In at least some embodiments, the input/output devices 158 may include one or more user-facing and device-facing interfaces associated with the receiver module 150. The input/output devices 158 may include a touch screen, a display, a monitor output, a USB-C port, another wired display port, a charging port, a data port, a microphone, a speaker, an audio interface, a camera interface, a button, a switch, a keyboard, a mouse, a haptic interface, and/or another control or output device. The input/output devices 158 may support delivery of endoscopic video to XR glasses or another display, receipt of user input, voice control, device pairing, setup, maintenance, recording control, and interaction with applications executed by the receiver module 150.

In at least some embodiments, the receiver module 150 may operate as a standalone device or as part of a distributed computing architecture in which one or more functions may be allocated among the video capture source, the receiver module 150, a docking device, an IXR box, a server, and/or a cloud-computing resource. Accordingly, any one or more of the processor 152, the algorithms and/or machine learning models 154, the memory 155, the communication interface and circuitry 157, and the input/output devices 158 may be consolidated in a single physical device or may be logically and/or physically distributed across multiple cooperating devices while still functioning together as the receiver module 150.

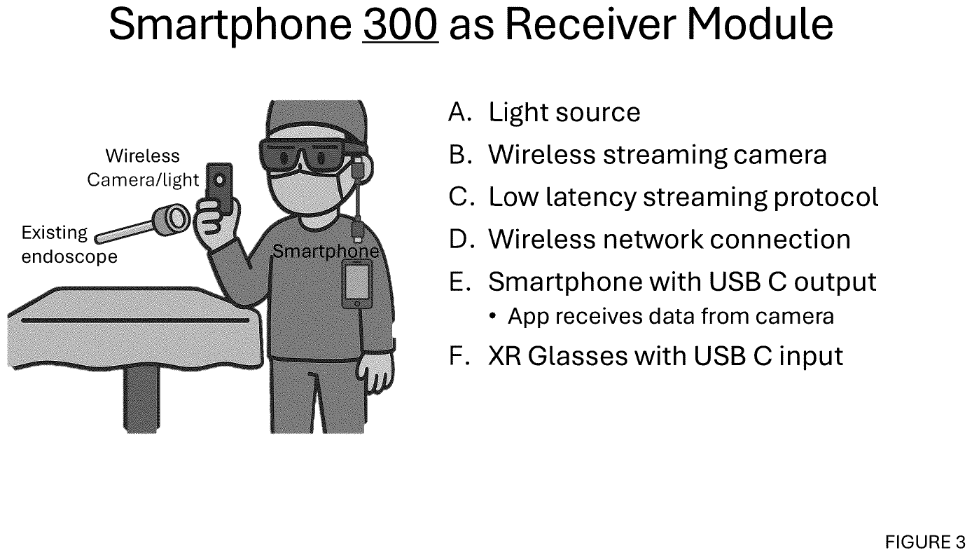

FIG. 3 is a schematic depicting a smartphone 300 as a receiver module in accordance with one or more embodiments of the present disclosure, where the system may include a light source A, a wireless streaming camera B, a low latency streaming protocol C, a wireless network connection D, a smartphone with USB-C output E, and XR glasses with USB-C input F.

In at least some embodiments, the light source A may include an illumination source associated with an existing endoscope, where the light source A may illuminate a body cavity or other anatomical region to facilitate image capture during a scope-based procedure.

In at least some embodiments, the wireless streaming camera B may include a video capture source coupled to the existing endoscope, where the wireless streaming camera B may capture endoscopic video and may transmit endoscopic video data from the existing endoscope toward the smartphone 300.

In at least some embodiments, the low latency streaming protocol C may include a streaming format and communication protocol selected to support live transmission of endoscopic video data with reduced delay, where the low latency streaming protocol C may support compressed transmission, uncompressed transmission, or both compressed transmission and uncompressed transmission.

In at least some embodiments, the wireless network connection D may include a wireless communication path between the wireless streaming camera B and the smartphone 300, where the wireless network connection D may carry the endoscopic video data transmitted in accordance with the low latency streaming protocol C.

In at least some embodiments, the smartphone with USB-C output E may include the receiver module of the system, where an application executed by the smartphone 300 may receive data from the wireless streaming camera B, may process the endoscopic video data, and may output video or output data through a USB-C interface.

In at least some embodiments, the smartphone 300 may be carried by a user during the procedure and may function as a portable receiver module, where the smartphone 300 may include at least one processor, memory, communication circuitry, and application software that may support reception, decoding, optional processing, and output of the endoscopic video data.

In at least some embodiments, the XR glasses with USB-C input F may include a head-mounted display configured to receive the output data from the smartphone 300 through a wired USB-C connection, where the XR glasses may present endoscopic video within a line of sight of the user during the scope-based procedure.

In at least some embodiments, FIG. 3 may illustrate an operational flow in which the light source A may illuminate the surgical site, the wireless streaming camera B may capture endoscopic video through the existing endoscope, the wireless streaming camera B may transmit the endoscopic video data using the low latency streaming protocol C over the wireless network connection D, the smartphone 300 may receive and may process the endoscopic video data through the application, and the smartphone 300 may output corresponding video or output data to the XR glasses F through the USB-C connection.

In at least some embodiments, the arrangement shown in FIG. 3 may provide a smartphone-based implementation of the receiver module portion of a modular endoscopic visualization platform, where the smartphone 300 may replace a dedicated receiver device while still enabling low-latency delivery of endoscopic video from a wireless camera/light arrangement to XR glasses.

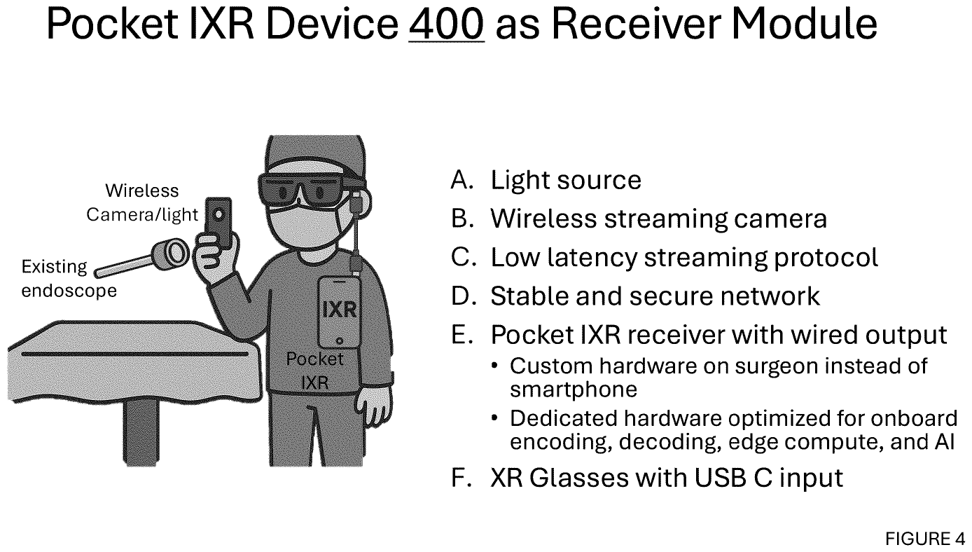

FIG. 4 is a schematic depicting a Pocket IXR device 400 as a receiver module in accordance with one or more embodiments of the present disclosure, where the system may include a light source A, a wireless streaming camera B, a low latency streaming protocol C, a stable and secure network D, a Pocket IXR receiver with wired output E, and XR glasses with USB-C input F.

In at least some embodiments, the light source A may include an illumination source configured to illuminate a body cavity or other anatomical region during a scope-based procedure, where the light source A may be associated with an endoscope used to provide visual access to the anatomical region.

In at least some embodiments, the wireless streaming camera B may include a video capture source coupled to the endoscope, where the wireless streaming camera B may capture endoscopic video and may transmit endoscopic video data from the endoscope toward the Pocket IXR receiver E.

In at least some embodiments, the low latency streaming protocol C may include a streaming format and communication protocol selected to support live transmission of the endoscopic video data with reduced delay, where the low latency streaming protocol C may support compressed transmission, uncompressed transmission, or both compressed transmission and uncompressed transmission.

In at least some embodiments, the stable and secure network D may include a wireless communication path over which the endoscopic video data may be transmitted from the wireless streaming camera B to the Pocket IXR receiver E, where the stable and secure network D may be configured to support bandwidth, reliability, and security appropriate for clinical use.

In at least some embodiments, the Pocket IXR receiver with wired output E may include a portable receiver module configured to receive the endoscopic video data over the stable and secure network D in accordance with the low latency streaming protocol C, may process the endoscopic video data, may decode the endoscopic video data when the endoscopic video data may be received in a compressed format, and may output video or output data over a wired connection.

In at least some embodiments, the Pocket IXR receiver E may include dedicated receiver-side hardware that may be optimized for onboard encoding, decoding, edge computing, and/or artificial intelligence processing, where the Pocket IXR receiver E may be wearable or otherwise portable so that the receiver module may be positioned on a user during the scope-based procedure.

In at least some embodiments, the XR glasses with USB-C input F may include a head-mounted display configured to receive the output data from the Pocket IXR receiver E through a wired USB-C connection, where the XR glasses may present endoscopic video within a line of sight of the user during the scope-based procedure.

In at least some embodiments, FIG. 4 may illustrate an operational flow in which the light source A may illuminate the surgical site, the wireless streaming camera B may capture endoscopic video through an endoscope, the wireless streaming camera B may transmit the endoscopic video data using the low latency streaming protocol C over the stable and secure network D, the Pocket IXR receiver E may receive and may process the endoscopic video data, and the Pocket IXR receiver E may output corresponding video or output data to the XR glasses F through the wired connection.

In at least some embodiments, the arrangement shown in FIG. 4 may provide a dedicated receiver-hardware implementation of the receiver module portion of the modular endoscopic visualization platform, where the Pocket IXR receiver E may provide a portable alternative to a smartphone-based receiver while supporting low-latency delivery of endoscopic video to XR glasses.

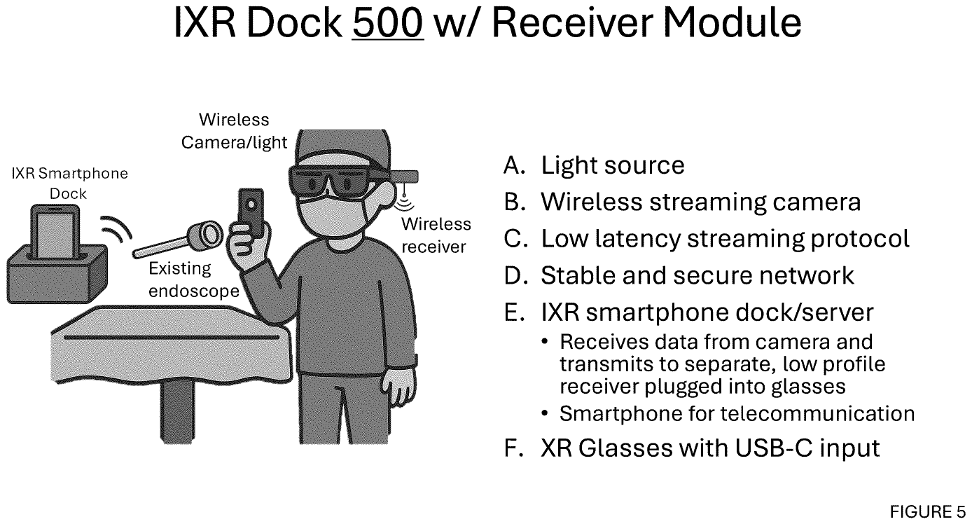

FIG. 5 is a schematic depicting an IXR smartphone dock/server implementation 500 with a separate receiver for XR glasses in accordance with one or more embodiments of the present disclosure, where the system may include a light source A, a wireless streaming camera B, a low latency streaming protocol C, a stable and secure network D, an IXR smartphone dock/server E, and XR glasses with a separate low-profile receiver F.

In at least some embodiments, the light source A may include an illumination source associated with an endoscope, where the light source A may illuminate a body cavity or other anatomical region during a scope-based procedure to facilitate capture of endoscopic video.

In at least some embodiments, the wireless streaming camera B may include a video capture source coupled to the endoscope, where the wireless streaming camera B may capture endoscopic video and may transmit endoscopic video data from the endoscope toward the IXR smartphone dock/server E.

In at least some embodiments, the low latency streaming protocol C may include a streaming format and communication protocol selected to support live transmission of the endoscopic video data with reduced delay, where the low latency streaming protocol C may support compressed transmission, uncompressed transmission, or both compressed transmission and uncompressed transmission.

In at least some embodiments, the stable and secure network D may include a wireless communication path between the wireless streaming camera B and the IXR smartphone dock/server E, where the stable and secure network D may support bandwidth, reliability, and security appropriate for clinical use.

In at least some embodiments, the IXR smartphone dock/server E may include a receiver-side device positioned off the sterile field, where the IXR smartphone dock/server E may receive the endoscopic video data from the wireless streaming camera B over the stable and secure network D in accordance with the low latency streaming protocol C, may process the endoscopic video data, and may transmit processed video or other output data toward the separate low-profile receiver associated with the XR glasses F.

In at least some embodiments, the IXR smartphone dock/server E may expand processing capability for edge computing and/or artificial intelligence processing beyond a pocket-type receiver device, where the IXR smartphone dock/server E may include additional compute resources, additional battery capacity, and/or additional communication resources enabled by a larger device located off the surgeon and off the sterile field.

In at least some embodiments, the IXR smartphone dock/server E may leverage smartphone functionality for telecommunication, networking, and/or related communication tasks, where receiver-side processing resources may be supplemented by the dock/server architecture while preserving a compact display-side hardware arrangement.

In at least some embodiments, the separate low-profile receiver associated with the XR glasses F may plug into the XR glasses and may receive transmitted output data from the IXR smartphone dock/server E, where the separate low-profile receiver may be kept as small as possible to reduce weight and bulk at the XR glasses.

In at least some embodiments, the XR glasses F may include a head-mounted display configured to present endoscopic video within a line of sight of the user during the scope-based procedure, where the XR glasses F may receive the output data through the separate receiver and may present the endoscopic video while allowing the larger processing hardware to remain off the user's head.

In at least some embodiments, FIG. 5 may illustrate an operational flow in which the light source A may illuminate the surgical site, the wireless streaming camera B may capture endoscopic video through the endoscope, the wireless streaming camera B may transmit the endoscopic video data using the low latency streaming protocol C over the stable and secure network D, the IXR smartphone dock/server E may receive and may process the endoscopic video data, and the IXR smartphone dock/server E may transmit corresponding output data to the separate low-profile receiver associated with the XR glasses F for presentation to the user.

In at least some embodiments, the arrangement shown in FIG. 5 may provide a hybrid receiver architecture in which higher-power receiver-side processing may be shifted to an off-body IXR smartphone dock/server E while a compact receiver may remain at the XR glasses F, where such architecture may support advanced edge computing and/or artificial intelligence functionality without requiring a larger receiver device to be worn by the user.

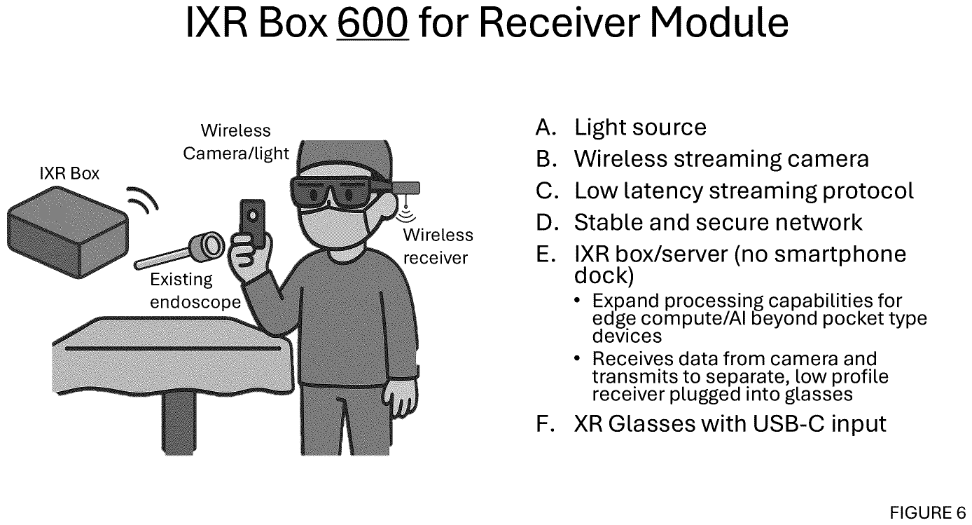

FIG. 6 is a schematic depicting an IXR box/server 600 receiver architecture in accordance with one or more embodiments of the present disclosure, where the system may include a light source A, a wireless streaming camera B, a low latency streaming protocol C, a stable and secure network D, an IXR box/server E, and XR glasses with USB-C input F.

In at least some embodiments, the light source A may include an illumination source associated with an existing endoscope, where the light source A may illuminate a body cavity or other anatomical region during a scope-based procedure to facilitate capture of endoscopic video.

In at least some embodiments, the wireless streaming camera B may include a video capture source coupled to the existing endoscope, where the wireless streaming camera B may capture endoscopic video and may transmit endoscopic video data from the endoscope toward the IXR box/server E.

In at least some embodiments, the low latency streaming protocol C may include a streaming format and communication protocol selected to support live transmission of the endoscopic video data with reduced delay, where the low latency streaming protocol C may support compressed transmission, uncompressed transmission, or both compressed transmission and uncompressed transmission.

In at least some embodiments, the stable and secure network D may include a wireless communication path between the wireless streaming camera B and the IXR box/server E, where the stable and secure network D may support bandwidth, reliability, and security appropriate for clinical use.

In at least some embodiments, the IXR box/server E may include a receiver-side device positioned off the user and/or off the sterile field, where the IXR box/server E may receive the endoscopic video data over the stable and secure network D in accordance with the low latency streaming protocol C, may process the endoscopic video data, and may output video or output data for presentation on the XR glasses F.

In at least some embodiments, the IXR box/server E may include at least one processor, memory, communication circuitry, and input/output hardware configured to support receiver functionality, edge computing, and/or transmission of the output data. The IXR box/server E may additionally or alternatively provide wireless access point functionality and/or local area network functionality for the visualization platform.

In at least some embodiments, the IXR box/server E may be implemented without a smartphone dock, where the IXR box/server E may function as a stand-alone receiver module and processing node for the endoscopic visualization platform.

In at least some embodiments, the XR glasses with USB-C input F may include a head-mounted display configured to receive the output data from the IXR box/server E through a wired USB-C connection, where the XR glasses F may present endoscopic video within a line of sight of the user during the scope-based procedure.

In at least some embodiments, FIG. 6 may illustrate an operational flow in which the light source A may illuminate the surgical site, the wireless streaming camera B may capture endoscopic video through the existing endoscope, the wireless streaming camera B may transmit the endoscopic video data using the low latency streaming protocol C over the stable and secure network D, the IXR box/server E may receive and may process the endoscopic video data, and the IXR box/server E may output corresponding video or output data to the XR glasses F for presentation to the user.

In at least some embodiments, the arrangement shown in FIG. 6 may provide a receiver architecture in which higher-power compute and networking functions may be located in the IXR box/server E rather than in a wearable receiver, where such architecture may support clinical deployment scenarios in which portability at the user may be balanced with more robust off-body processing and communication resources.

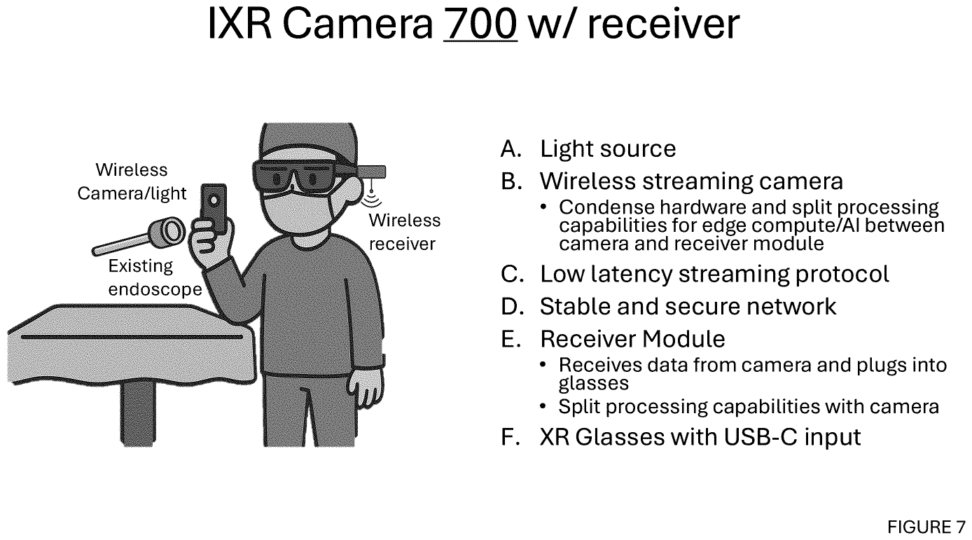

FIG. 7 is a schematic depicting an IXR camera 700 with receiver system in accordance with one or more embodiments of the present disclosure, where the system may include a light source A, a wireless streaming camera B, a low latency streaming protocol C, a stable and secure network D, a receiver module E, and XR glasses with USB-C input F.

In at least some embodiments, the light source A may include an illumination source associated with an endoscope, where the light source A may illuminate a body cavity or other anatomical region during a scope-based procedure to facilitate capture of endoscopic video.

In at least some embodiments, the wireless streaming camera B may include a video capture source coupled to the endoscope, where the wireless streaming camera B may capture endoscopic video and may transmit endoscopic video data from the endoscope toward the receiver module E. In some implementations, the wireless streaming camera B may additionally include at least a portion of receiver-side functionality, processing functionality, encoding functionality, decoding functionality, edge computing functionality, and/or artificial intelligence functionality.

In at least some embodiments, the low latency streaming protocol C may include a streaming format and communication protocol selected to support live transmission of the endoscopic video data with reduced delay, where the low latency streaming protocol C may support compressed transmission, uncompressed transmission, or both compressed transmission and uncompressed transmission.

In at least some embodiments, the stable and secure network D may include a wireless communication path between the wireless streaming camera B and the receiver module E, where the stable and secure network D may support bandwidth, reliability, and security appropriate for clinical use.

In at least some embodiments, the receiver module E may include a receiver-side device configured to receive the endoscopic video data over the stable and secure network D in accordance with the low latency streaming protocol C, may process the endoscopic video data, may decode the endoscopic video data when the endoscopic video data may be received in a compressed format, and may output video or output data for presentation on the XR glasses F. The receiver module E may include a smartphone, a Pocket IXR device, a docked device, a compact receiver, and/or another receiver-side hardware implementation.

In at least some embodiments, receiver-side functionality may be distributed between the wireless streaming camera B and the receiver module E, where the wireless streaming camera B may perform a first portion of processing associated with at least one of encoding, decoding, edge computing, or artificial intelligence processing, and where the receiver module E may perform a second portion of processing before output to the XR glasses F.

In at least some embodiments, the wireless streaming camera B and the receiver module E together may define an IXR camera with receiver system in which compute, communication, and display-delivery responsibilities may be partitioned across camera-side hardware and receiver-side hardware based on latency, power, thermal, ergonomic, and/or workflow considerations.

In at least some embodiments, the XR glasses with USB-C input F may include a head-mounted display configured to receive the output data from the receiver module E through a wired USB-C connection, where the XR glasses F may present endoscopic video within a line of sight of the user during the scope-based procedure.

In at least some embodiments, FIG. 7 may illustrate an operational flow in which the light source A may illuminate the surgical site, the wireless streaming camera B may capture endoscopic video through the endoscope, the wireless streaming camera B may transmit the endoscopic video data using the low latency streaming protocol C over the stable and secure network D, the receiver module E may receive and may further process the endoscopic video data, and the receiver module E may output corresponding video or output data to the XR glasses F for presentation to the user.

In at least some embodiments, the arrangement shown in FIG. 7 may provide a modular architecture in which at least some receiver functionality may be shifted toward the wireless streaming camera B while preserving a separate receiver module E for downstream output to the XR glasses F, where such architecture may support a reduced-size receiver, a reduced processing burden at the receiver module E, and/or a selected allocation of processing resources across the system.

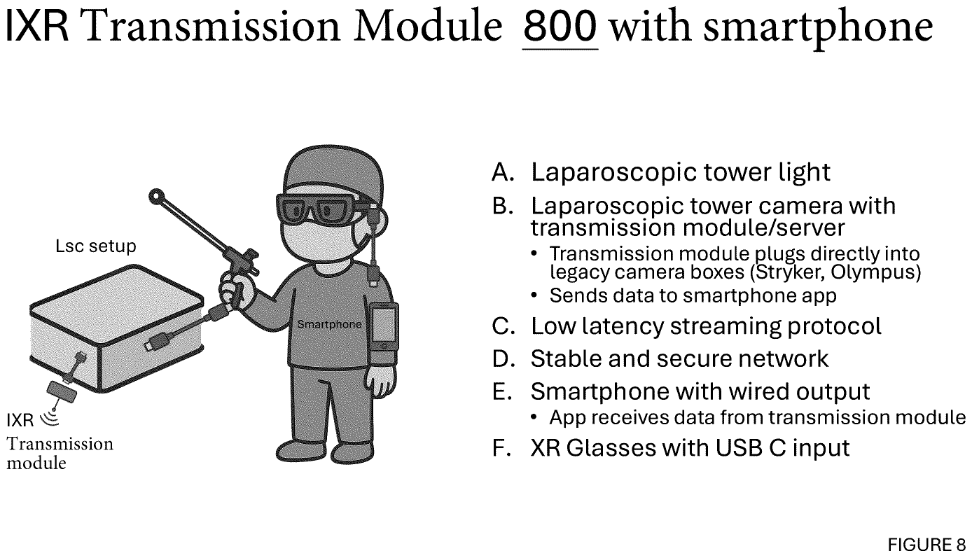

FIG. 8 is a schematic depicting an IXR transmission module 800 visualization architecture with a smartphone receiver in accordance with one or more embodiments of the present disclosure, where the system may include a legacy laparoscopic tower light A, a legacy laparoscopic tower camera with a transmission module/server B, a low latency streaming protocol C, a stable and secure network D, a smartphone with wired output E, and XR glasses with USB-C input F.

In at least some embodiments, the legacy laparoscopic tower light A may include a light source of a legacy laparoscopy setup, where the legacy laparoscopic tower light A may illuminate a body cavity through an endoscope during a scope-based procedure.

In at least some embodiments, the legacy laparoscopic tower camera with the transmission module/server B may include a video capture source associated with the legacy laparoscopy setup, where the legacy laparoscopic tower camera may receive optical information from the endoscope and may generate endoscopic video data. The transmission module/server B may plug directly into a legacy camera box and may create a communication path for sending the endoscopic video data away from the tower environment.

In at least some embodiments, the transmission module/server B may include an IXR transmission module that may interface with legacy laparoscopic tower equipment and may transmit the endoscopic video data from the legacy laparoscopic tower camera toward the smartphone E. The IXR transmission module may permit the visualization platform to interoperate with existing tower hardware without replacing the legacy camera system.

In at least some embodiments, the low latency streaming protocol C may include a streaming format and communication protocol selected to support live transmission of the endoscopic video data with reduced delay, where the low latency streaming protocol C may support compressed transmission, uncompressed transmission, or both compressed transmission and uncompressed transmission.

In at least some embodiments, the stable and secure network D may include a wireless communication path established by or used with the transmission module/server B, where the stable and secure network D may carry the endoscopic video data from the transmission module/server B to the smartphone E.

In at least some embodiments, the smartphone with wired output E may include a receiver module executing an application configured to receive the endoscopic video data from the transmission module/server B, may process the endoscopic video data, and may output video or output data through a wired interface. The application executed by the smartphone E may function as a viewer application for the endoscopic video data received from the IXR transmission module.

In at least some embodiments, the smartphone E may receive the endoscopic video data over the stable and secure network D in accordance with the low latency streaming protocol C and may output corresponding video or output data over a wired connection to the XR glasses F. The wired output of the smartphone E may include a USB-C output or another suitable wired display interface.

In at least some embodiments, the XR glasses with USB-C input F may include a head-mounted display configured to receive the output data from the smartphone E and to present endoscopic video within a line of sight of the user during the scope-based procedure.

In at least some embodiments, FIG. 8 may illustrate an operational flow in which the legacy laparoscopic tower light A may illuminate the surgical site, the legacy laparoscopic tower camera and transmission module/server B may acquire and forward endoscopic video data from the legacy laparoscopy setup, the low latency streaming protocol C and the stable and secure network D may deliver the endoscopic video data to the smartphone E, the application on the smartphone E may receive and may process the endoscopic video data, and the smartphone E may output corresponding video or output data to the XR glasses F for presentation to the user.

In at least some embodiments, the arrangement shown in FIG. 8 may provide a visualization-only retrofit for a legacy laparoscopic tower environment, where the IXR transmission module may plug into existing legacy camera boxes and may send video to the smartphone E for visualization through the connected XR glasses F, thereby permitting head-mounted display use without replacing the legacy laparoscopic tower camera and light infrastructure.

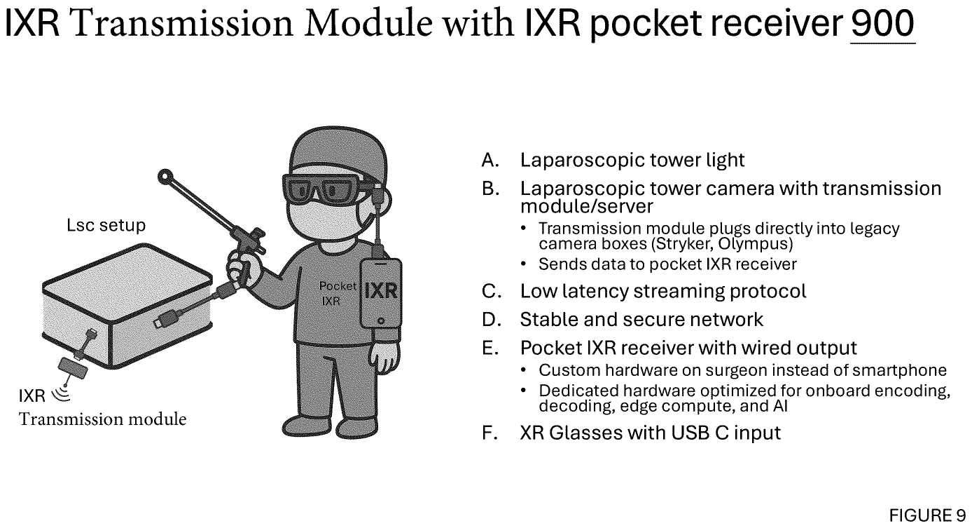

FIG. 9 is a schematic depicting an IXR transmission module visualization architecture with a Pocket IXR receiver 900 in accordance with one or more embodiments of the present disclosure, where the system may include a laparoscopic tower light A, a laparoscopic tower camera with a transmission module/server B, a low latency streaming protocol C, a stable and secure network D, a Pocket IXR receiver with wired output E, and XR glasses with USB-C input F.

In at least some embodiments, the laparoscopic tower light A may include a light source of a legacy laparoscopy setup, where the laparoscopic tower light A may illuminate a body cavity through an endoscope during a scope-based procedure.

In at least some embodiments, the laparoscopic tower camera with the transmission module/server B may include a video capture source associated with the legacy laparoscopy setup, where the laparoscopic tower camera may receive optical information from the endoscope and may generate endoscopic video data. The transmission module/server B may plug directly into a legacy camera box and may create a communication path for sending the endoscopic video data away from the tower environment.

In at least some embodiments, the transmission module/server B may include an IXR transmission module that may interface with legacy laparoscopic tower equipment and may transmit the endoscopic video data from the laparoscopic tower camera toward the Pocket IXR receiver E. The IXR transmission module may permit the visualization platform to interoperate with existing tower hardware without replacing the legacy camera system.

In at least some embodiments, the low latency streaming protocol C may include a streaming format and communication protocol selected to support live transmission of the endoscopic video data with reduced delay, where the low latency streaming protocol C may support compressed transmission, uncompressed transmission, or both compressed transmission and uncompressed transmission.

In at least some embodiments, the stable and secure network D may include a wireless communication path established by or used with the transmission module/server B, where the stable and secure network D may carry the endoscopic video data from the transmission module/server B to the Pocket IXR receiver E.

In at least some embodiments, the Pocket IXR receiver with wired output E may include a dedicated receiver module configured to receive the endoscopic video data from the transmission module/server B, may process the endoscopic video data, and may output video or output data through a wired interface. The Pocket IXR receiver E may include onboard hardware optimized for low-latency video handling and may support decoding, edge computing, and/or artificial intelligence processing.

In at least some embodiments, the Pocket IXR receiver E may receive the endoscopic video data over the stable and secure network D in accordance with the low latency streaming protocol C and may output corresponding video or output data over a wired connection to the XR glasses F. The wired output of the Pocket IXR receiver E may include a USB-C output or another suitable wired display interface.

In at least some embodiments, the XR glasses with USB-C input F may include a head-mounted display configured to receive the output data from the Pocket IXR receiver E and to present endoscopic video within a line of sight of the user during the scope-based procedure.

In at least some embodiments, FIG. 9 may illustrate an operational flow in which the laparoscopic tower light A may illuminate the surgical site, the laparoscopic tower camera and transmission module/server B may acquire and forward endoscopic video data from the legacy laparoscopy setup, the low latency streaming protocol C and the stable and secure network D may deliver the endoscopic video data to the Pocket IXR receiver E, the Pocket IXR receiver E may receive and may process the endoscopic video data, and the Pocket IXR receiver E may output corresponding video or output data to the XR glasses F for presentation to the user.

In at least some embodiments, the arrangement shown in FIG. 9 may provide a visualization-only retrofit for a legacy laparoscopic tower environment, where the IXR transmission module may plug into existing legacy camera boxes and may send video to the Pocket IXR receiver E for visualization through the connected XR glasses F, thereby permitting head-mounted display use without replacing the legacy laparoscopic tower camera and light infrastructure.

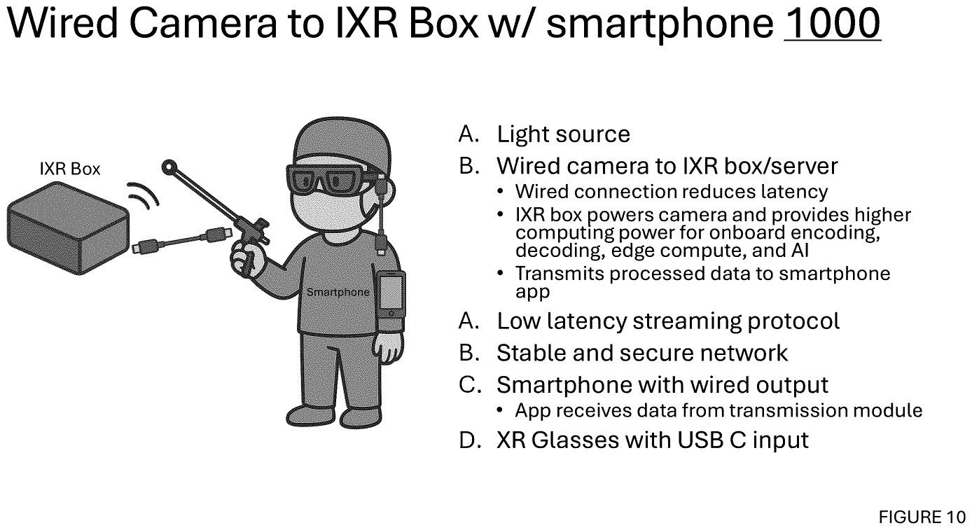

FIG. 10 is a schematic depicting an IXR box setup with a smartphone 1000 in accordance with one or more embodiments of the present disclosure, where the system may include a light source A, a wired camera to IXR box/server B, a low latency streaming protocol C, a stable and secure network D, a smartphone with wired output E, and XR glasses with USB-C input F.

In at least some embodiments, the light source A may include an illumination source associated with an endoscope, where the light source A may illuminate a body cavity or other anatomical region during a scope-based procedure to facilitate image capture.

In at least some embodiments, the wired camera to IXR box/server B may include a video capture source coupled to the endoscope and physically connected to an IXR box/server, where the wired connection may reduce latency and may provide a direct path for transmission of endoscopic video data from the camera to the IXR box/server. In at least some implementations, the wired connection may additionally provide power and/or data connectivity between the camera and the IXR box/server.

In at least some embodiments, the IXR box/server associated with element B may receive endoscopic video data from the wired camera and may function as a receiver-side processing node, where the IXR box/server may process the endoscopic video data and may prepare video or output data for downstream delivery to the smartphone E.

In at least some embodiments, the low latency streaming protocol C may include a streaming format and communication protocol selected to support live transmission of the endoscopic video data with reduced delay, where the low latency streaming protocol C may support compressed transmission, uncompressed transmission, or both compressed transmission and uncompressed transmission for communication downstream of the IXR box/server.

In at least some embodiments, the stable and secure network D may include a communication path between the IXR box/server and the smartphone E, where the stable and secure network D may carry the endoscopic video data and/or processed output data from the IXR box/server to the smartphone E.

In at least some embodiments, the smartphone with wired output E may include a receiver-side display interface device configured to receive the endoscopic video data and/or processed output data from the IXR box/server over the stable and secure network D and to output corresponding video or output data through a wired connection. The smartphone E may execute an application that may display, relay, and/or manage the data for presentation on the XR glasses F.

In at least some embodiments, the XR glasses with USB-C input F may include a head-mounted display configured to receive the output data from the smartphone E through a wired USB-C connection, where the XR glasses F may present endoscopic video within a line of sight of the user during the scope-based procedure.

In at least some embodiments, FIG. 10 may illustrate an operational flow in which the light source A may illuminate the surgical site, the wired camera to IXR box/server B may capture endoscopic video through the endoscope and may send the endoscopic video data to the IXR box/server over a wired connection, the IXR box/server may use the low latency streaming protocol C and the stable and secure network D to deliver the endoscopic video data and/or processed output data to the smartphone E, and the smartphone E may output corresponding video or output data to the XR glasses F for presentation to the user.

In at least some embodiments, the arrangement shown in FIG. 10 may provide a hybrid architecture in which a wired upstream connection between the camera and the IXR box/server may reduce latency and may centralize compute and networking resources at the IXR box/server, while the smartphone E may function as a downstream receiver and display-output interface for the XR glasses F.

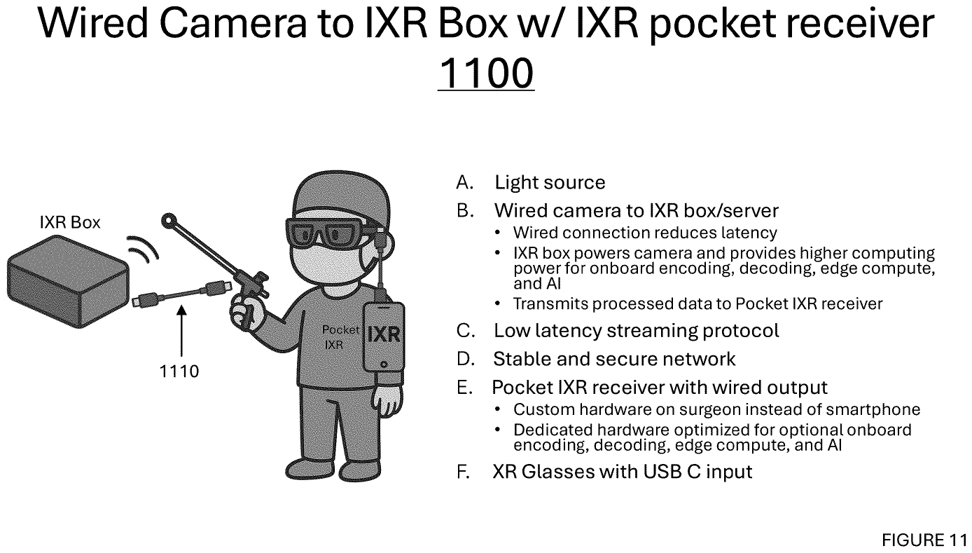

FIG. 11 is a schematic depicting an a wired camera to IXR box setup with an IXR pocket receiver 1100 in accordance with one or more embodiments of the present disclosure, where the system may include a light source A, a wired camera to IXR box/server B via a wired connect 1110, a low latency streaming protocol C, a stable and secure network D, a Pocket IXR receiver with wired output E, and XR glasses with USB-C input F.

In at least some embodiments, the light source A may include an illumination source associated with an endoscope, where the light source A may illuminate a body cavity or other anatomical region during a scope-based procedure to facilitate image capture.

In at least some embodiments, the wired camera to IXR box/server B may include a video capture source coupled to the endoscope and physically connected to an IXR box/server, where the wired connection 1110 may reduce latency and may provide a direct path for transmission of endoscopic video data from the camera to the IXR box/server. In at least some implementations, the wired connection 1110 may additionally provide power and/or data connectivity between the camera and the IXR box/server.

In at least some embodiments, the IXR box/server associated with element B may receive endoscopic video data from the wired camera and may function as a receiver-side processing node, where the IXR box/server may process the endoscopic video data and may prepare video or output data for downstream delivery to the Pocket IXR receiver E.

In at least some embodiments, the low latency streaming protocol C may include a streaming format and communication protocol selected to support live transmission of the endoscopic video data with reduced delay, where the low latency streaming protocol C may support compressed transmission, uncompressed transmission, or both compressed transmission and uncompressed transmission for communication downstream of the IXR box/server.

In at least some embodiments, the stable and secure network D may include a communication path between the IXR box/server and the Pocket IXR receiver E, where the stable and secure network D may carry the endoscopic video data and/or processed output data from the IXR box/server to the Pocket IXR receiver E.

In at least some embodiments, the Pocket IXR receiver with wired output E may include a portable receiver module configured to receive the endoscopic video data and/or processed output data from the IXR box/server over the stable and secure network D and to output corresponding video or output data through a wired connection. The Pocket IXR receiver E may include onboard hardware optimized for low-latency video handling and may support decoding, edge computing, and/or artificial intelligence processing.

In at least some embodiments, the Pocket IXR receiver E may be wearable or otherwise portable so that the receiver module may be positioned on a user during the scope-based procedure, where the Pocket IXR receiver E may function as a downstream receiver and display-output interface between the IXR box/server and the XR glasses F.

In at least some embodiments, the XR glasses with USB-C input F may include a head-mounted display configured to receive the output data from the Pocket IXR receiver E through a wired USB-C connection, where the XR glasses F may present endoscopic video within a line of sight of the user during the scope-based procedure.

In at least some embodiments, FIG. 11 may illustrate an operational flow in which the light source A may illuminate the surgical site, the wired camera to IXR box/server B may capture endoscopic video through the endoscope and may send the endoscopic video data to the IXR box/server over a wired connection 1110, the IXR box/server may use the low latency streaming protocol C and the stable and secure network D to deliver the endoscopic video data and/or processed output data to the Pocket IXR receiver E, and the Pocket IXR receiver E may output corresponding video or output data to the XR glasses F for presentation to the user.

In at least some embodiments, the arrangement shown in FIG. 11 may provide a hybrid architecture in which a wired upstream connection between the camera and the IXR box/server may reduce latency and may centralize compute and networking resources at the IXR box/server, while the Pocket IXR receiver E may function as a portable downstream receiver for delivering the output data to the XR glasses F.

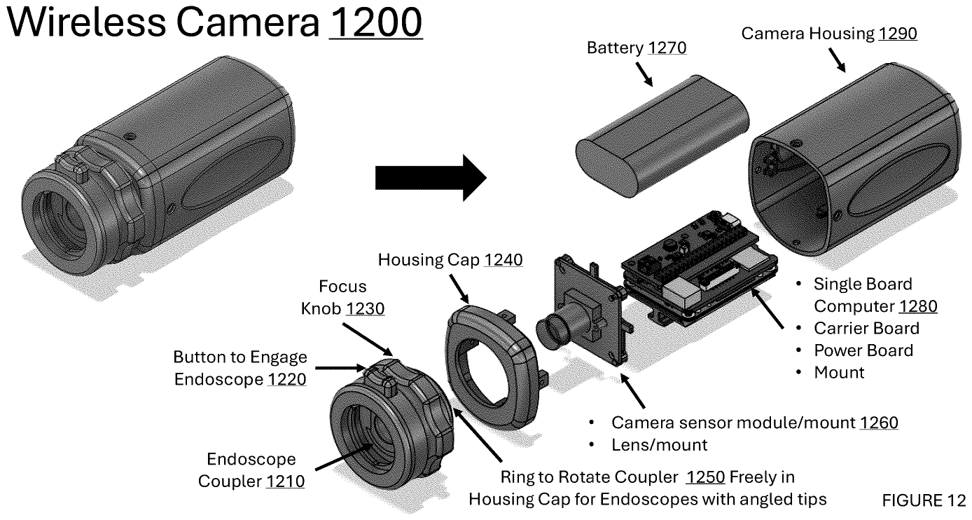

FIG. 12 is an exploded view depicting a wireless camera 1200 in accordance with one or more embodiments of the present disclosure, where the wireless camera 1200 may include a camera housing 1290, a battery 1270, a housing cap 1240, a focus knob 1230, a button 1220 to engage an endoscope, an endoscope coupler 1210, a ring 1250 to rotate the coupler freely in the housing cap for endoscopes with angled tips, a camera sensor module and/or mount 1260, a single board computer 1280, a carrier board, a power board, and a mount.

In at least some embodiments, the wireless camera 1200 may include a video capture source configured to attach to an endoscope and to acquire endoscopic video for transmission to a receiver module, where the wireless camera 1200 may be configured for use in a portable endoscopic visualization platform employing low-latency streaming to a head-mounted display.

In at least some embodiments, the camera housing 1290 may support and enclose internal components of the wireless camera 1200, where the camera housing 1290 may define an external body sized and shaped for handling, attachment, and use during a scope-based procedure.

In at least some embodiments, the housing cap 1240 may close at least a portion of the camera housing 1290 and may support mounting and alignment of endoscope-coupling components, where the housing cap 1240 may retain the endoscope coupler and associated rotation features.

In at least some embodiments, the endoscope coupler 1210 may mechanically interface with an endoscope to couple the wireless camera 1200 to the endoscope, where the endoscope coupler 1210 may align the optical path of the endoscope with the image capture components of the wireless camera 1200.

In at least some embodiments, the button 1220 to engage the endoscope may actuate a locking or retention mechanism associated with the endoscope coupler 1210, where the button 1220 may permit the wireless camera 1200 to be selectively attached to or detached from the endoscope.

In at least some embodiments, the ring 1250 may permit the endoscope coupler to rotate freely relative to the housing cap, where such rotational freedom may facilitate positioning and alignment of the wireless camera 1200 for endoscopes having angled tips.

In at least some embodiments, the focus knob 1230 may provide manual focus adjustment for the wireless camera 1200, where rotation of the focus knob 1230 may adjust optical focus associated with the camera sensor module and/or mount 1260 to improve image quality while the wireless camera 1200 may remain attached to the endoscope.

In at least some embodiments, the camera sensor module and/or mount 1260 may include an image sensor and supporting structure for optical alignment, where the camera sensor module and/or mount 1260 may receive image information from the endoscope and may generate image data corresponding to the endoscopic field of view.

In at least some embodiments, the single board computer 1280 may include processing circuitry configured to control operation of the wireless camera 1200, where the single board computer 1280 may support image acquisition, video handling, communication control, and/or transmission of endoscopic video data.

In at least some embodiments, the carrier board may electrically and mechanically support one or more components of the wireless camera 1200, where the carrier board may provide interconnection between the single board computer 1280, the camera sensor module and/or mount 1260, and other electronic components.

In at least some embodiments, the power board may manage power distribution within the wireless camera 1200, where the power board may distribute electrical power from the battery 1270 to the single board computer 1280, the camera sensor module and/or mount 1260, and other powered elements of the wireless camera 1200.

In at least some embodiments, the battery 1270 may provide onboard electrical power for the wireless camera 1200, where the battery 1270 may support portable use of the wireless camera 1200 without requiring a continuous external power cable during image capture and wireless transmission.

In at least some embodiments, the mount may support positioning and retention of one or more internal components within the camera housing 1290, where the mount may facilitate assembly and alignment of the camera sensor module and/or mount 1260, the single board computer 1280, the carrier board, and the power board.

In at least some embodiments, FIG. 12 may illustrate an arrangement in which optical, mechanical, power, and compute elements may be integrated into the wireless camera 1200 to provide a compact video capture source attachable to an endoscope, where the wireless camera 1200 may capture endoscopic video, may generate endoscopic video data, and may transmit the endoscopic video data to a separate receiver module for presentation on a head-mounted display.

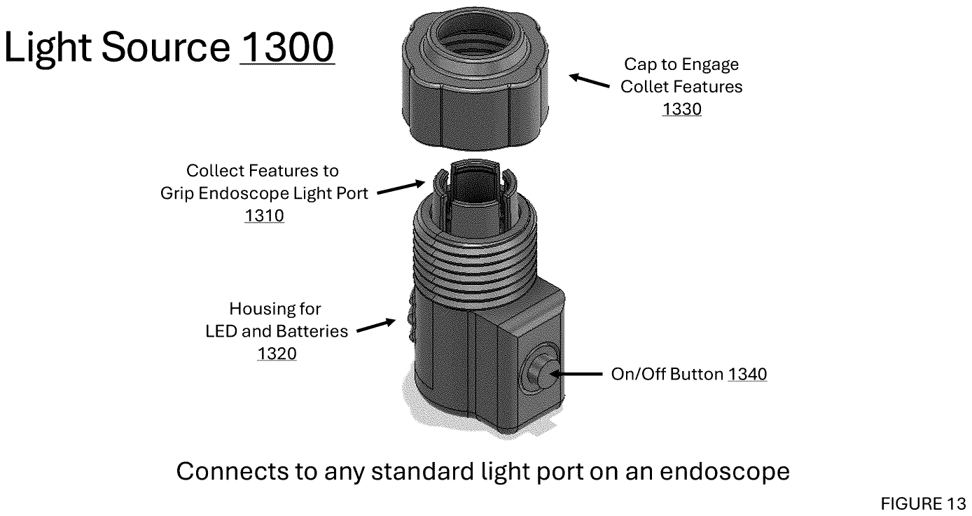

FIG. 13 is an exploded view depicting a light source assembly 1300 for an endoscope in accordance with one or more embodiments of the present disclosure, where the light source assembly 1300 may include collet features 1310 to grip an endoscope light port, a housing 1320 for an LED and batteries, a cap 1330 to engage the collet features, and an on/off button 1340.

In at least some embodiments, the light source assembly 1300 may include a portable illumination module configured to attach to an endoscope and to illuminate a body cavity or other anatomical region during a scope-based procedure, where the light source assembly may reduce reliance on larger external light infrastructure.

In at least some embodiments, the collet features 1310 may engage an endoscope light port to mechanically secure the light source assembly 1300 to the endoscope, where the collet features 1310 may provide a gripping interface that may support stable attachment during handling and use.

In at least some embodiments, the housing 1320 may retain an LED and one or more batteries, where the LED may generate illumination for delivery through the endoscope and where the one or more batteries may provide onboard electrical power for the light source assembly.

In at least some embodiments, the housing 1320 may support a compact and portable light source arrangement, where the housing 1320 may position the LED relative to the endoscope light port and may support electrical and mechanical integration of the internal components.

In at least some embodiments, the cap 1330 may engage the collet features 1310 to retain the light source assembly on the endoscope light port, where the cap 1330 may cooperate with the collet features 1310 to tighten, secure, and/or stabilize the attached condition of the light source assembly.

In at least some embodiments, the on/off button 1340 may control delivery of power from the one or more batteries to the LED, where actuation of the on/off button 1340 may selectively activate and deactivate illumination.

In at least some embodiments, FIG. 13 may illustrate an arrangement in which the collet features 1310, the housing 1320, the cap 1330, and the on/off button 1340 may cooperate to provide a self-contained light source assembly 1300 that may attach directly to the endoscope, may provide illumination during image capture, and may support use of the endoscopic visualization platform in a portable clinical workflow.

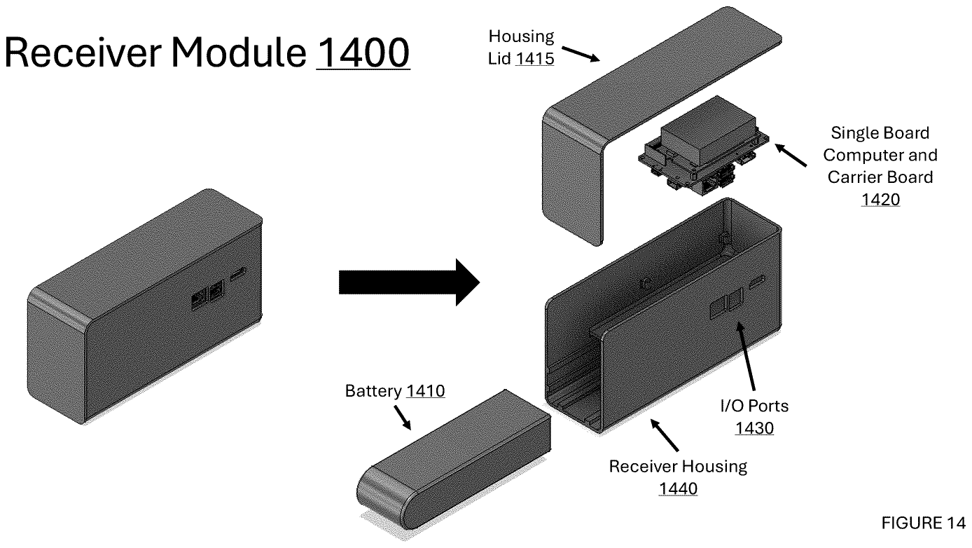

FIG. 14 is an exploded view depicting a receiver module 1400 in accordance with one or more embodiments of the present disclosure, where the receiver module 1400 may include a housing lid 1415, a battery 1410, a single board computer and carrier board 1420, input/output ports 1430, and a receiver housing 1440.

In at least some embodiments, the receiver module 1400 may include a portable receiver-side device configured to receive endoscopic video data from a video capture source over a network connection in accordance with a streaming protocol and to output video or output data for presentation on a head-mounted display.

In at least some embodiments, the receiver housing 1440 may support and enclose internal components of the receiver module 1400, where the receiver housing 1440 may define an external body sized and shaped for portable use during a scope-based procedure.

In at least some embodiments, the housing lid 1415 may close at least a portion of the receiver housing 1440 and may retain internal components within the receiver module 1400, where the housing lid 1415 may facilitate assembly and access to internal hardware.

In at least some embodiments, the battery 1410 may provide onboard electrical power for the receiver module 1400, where the battery 1410 may support portable and wearable use of the receiver module 1400 without requiring a continuous external power source during reception and processing of endoscopic video data.

In at least some embodiments, the single board computer and carrier board 1420 may include processing circuitry and supporting electronics configured to control operation of the receiver module 1400, where the single board computer and carrier board 1420 may support reception of endoscopic video data, decoding of the endoscopic video data when the endoscopic video data may be received in a compressed format, optional edge computing and/or artificial intelligence processing, generation of output data, and communication with external devices.

In at least some embodiments, the input/output ports 1430 may provide electrical and data interfaces for the receiver module 1400, where the input/output ports 1430 may include one or more wired output ports for connection to XR glasses and may additionally or alternatively include charging ports, data ports, and/or ports for communication with other system components.

In at least some embodiments, the receiver module 1400 may be implemented as a Pocket IXR device or another portable receiver module configuration, where the arrangement of the housing lid 1415, the battery 1410, the single board computer and carrier board 1420, the input/output ports 1430, and the receiver housing 1440 may support a compact architecture for receiving, processing, and outputting endoscopic video data.

In at least some embodiments, FIG. 14 may illustrate an arrangement in which power, compute, interface, and enclosure elements may be integrated into the receiver module 1400 to provide a self-contained receiver-side device that may cooperate with a separate video capture source and may deliver output data to a head-mounted display within a line of sight of a user during a scope-based procedure.

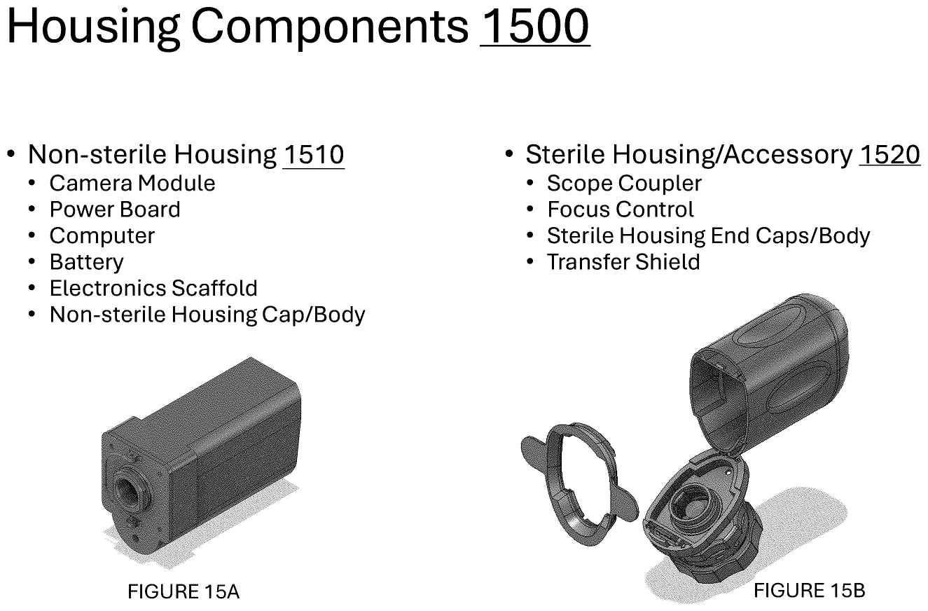

FIGS. 15A and 15B are views of a housing architecture 1500 in accordance with one or more embodiments of the present disclosure, where FIG. 15A may identify principal components of the housing architecture 1500 and FIG. 15B may depict at least a portion of a sterile housing/accessory 1520 in an exploded arrangement. In at least some embodiments, the housing architecture 1500 may include a non-sterile housing 1510 and a sterile housing/accessory 1520.

In at least some embodiments, the non-sterile housing 1510 may include a camera module, a power board, a programmable single-board computer (SBC) with wireless communication capabilities and general-purpose input/output (GPIO) pins, a battery, an electronics scaffold, and a non-sterile housing cap/body. The non-sterile housing 1510 may contain active electronic components of a camera assembly and may provide a structural enclosure for such components during use.

In at least some embodiments, the camera module may capture endoscopic video from an endoscope, where the camera module may be optically aligned with the endoscope and may generate image data for processing and transmission. The power board may distribute electrical power among the electronic components of the non-sterile housing 1510, and the computer may control image acquisition, device operation, and communication functions associated with the camera module.

In at least some embodiments, the battery may provide onboard electrical power for the non-sterile housing 1510, where the battery may support portable operation without requiring a continuous external power cable. The electronics scaffold may support and position the camera module, the power board, the computer, and the battery within the non-sterile housing cap/body.

In at least some embodiments, the sterile housing/accessory 1520 may include a scope coupler, a focus control, sterile housing end caps/body, and a transfer shield. The sterile housing/accessory 1520 may receive at least a portion of the non-sterile housing 1510 so that a sterile-contact exterior may be presented during a procedure while the active electronics may remain enclosed within the non-sterile housing 1510.

In at least some embodiments, the scope coupler may interface mechanically with an endoscope and may align an optical pathway of the endoscope with the camera module contained in the non-sterile housing 1510. The focus control may permit adjustment of optical focus while the housing architecture 1500 may remain attached to the endoscope.

In at least some embodiments, the sterile housing end caps/body may define an outer sterile enclosure around the non-sterile housing 1510, where the sterile housing end caps/body may support assembly, closure, and use of the housing architecture 1500 in a sterile field. The transfer shield may facilitate transfer of the non-sterile housing 1510 into the sterile housing/accessory 1520 and may reduce contamination risk during assembly.

In at least some embodiments, FIG. 15B may depict the sterile housing/accessory 1520 in a separated condition, where individual parts of the sterile housing/accessory 1520 may be assembled around the non-sterile housing 1510. In this manner, the housing architecture 1500 may separate non-sterile electronic components from sterile-contact surfaces while still permitting optical coupling to the endoscope and focus adjustment during use.

In at least some embodiments, the housing architecture 1500 shown in FIGS. 15A and 15B may address sterilization and thermal-management considerations by locating heat-generating and power-consuming electronics in the non-sterile housing 1510 while using the sterile housing/accessory 1520 to provide a clinically acceptable outer interface for handling and use during a scope-based procedure.

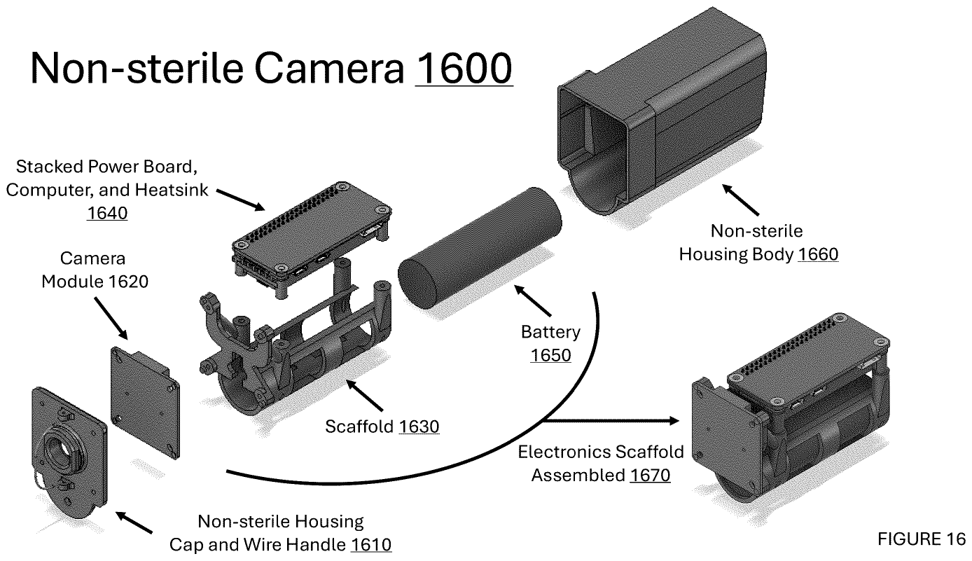

FIG. 16 is an exploded view depicting a non-sterile camera 1600 in accordance with one or more embodiments of the present disclosure, where the non-sterile camera 1600 may include a non-sterile housing cap and wire handle 1610, a camera module 1620, a scaffold 1630, a stacked power board, computer, and heatsink 1640, a battery 1650, a non-sterile housing body 1660, and an electronics scaffold assembled 1670.

In at least some embodiments, the non-sterile camera 1600 may include a camera assembly configured to capture endoscopic video for transmission to a receiver module, where the non-sterile camera 1600 may house active electronic components in a non-sterile enclosure that may later be used with a sterile housing arrangement.

In at least some embodiments, the non-sterile housing cap and wire handle 1610 may define an upper enclosure portion of the non-sterile camera 1600, where the non-sterile housing cap may retain internal components and the wire handle may facilitate handling, transfer, insertion into a sterile housing, and/or assembly operations.

In at least some embodiments, the wire handle of the non-sterile camera assembly may be movable between a raised position used for handling and insertion and a lowered position in which the wire handle may lie substantially flush against a cap or housing portion of the non-sterile camera assembly, where movement of the wire handle to the lowered position may reduce interference with closure of the sterile housing assembly 1700. In at least some embodiments, the wire handle may be coupled to the non-sterile camera assembly by a hinge, pivot, resilient joint, living hinge structure, deformable wire segment, or other movable attachment that may permit repeated raising and lowering of the wire handle.

In at least some embodiments, the wire handle may be biased toward the lowered position, may be retained in the lowered position by friction, detent engagement, snap engagement, interference fit, magnetic retention, elastic retention, and/or another holding arrangement, or any combination thereof, where the wire handle may remain substantially flush during closure of the sterile housing cap 1740 and during use of the assembled device. In at least some embodiments, retention of the wire handle in the lowered position may facilitate enclosure of the non-sterile camera assembly within the sterile housing assembly 1700 while reducing unintended movement of the wire handle during transfer and sealing.

In at least some embodiments, the camera module 1620 may capture endoscopic video from an endoscope, where the camera module 1620 may be optically aligned relative to an endoscope coupler and may generate image data corresponding to an endoscopic field of view.

In at least some embodiments, the scaffold 1630 may support positioning and alignment of internal components within the non-sterile camera 1600, where the scaffold 1630 may maintain spatial relationships among the camera module 1620, the stacked power board, computer, and heatsink 1640, and the battery 1650.

In at least some embodiments, the stacked power board, computer, and heatsink 1640 may include a power board, a computing device, and a thermal management element arranged in a stacked configuration, where the power board may distribute electrical power, the computer may control image acquisition and communication functions, and the heatsink may dissipate heat generated during operation.

In at least some embodiments, the computer within the stacked power board, computer, and heatsink 1640 may control operation of the non-sterile camera 1600, may manage image acquisition from the camera module 1620, and may support communication and/or transmission of endoscopic video data to a receiver module.

In at least some embodiments, the battery 1650 may provide onboard electrical power for the non-sterile camera 1600, where the battery 1650 may support portable operation of the camera assembly without requiring a continuous external power cable during image capture and transmission.

In at least some embodiments, the non-sterile housing body 1660 may define a lower enclosure portion of the non-sterile camera 1600, where the non-sterile housing body 1660 may cooperate with the non-sterile housing cap and wire handle 1610 to enclose and protect the internal components of the non-sterile camera 1600.

In at least some embodiments, the electronics scaffold assembled 1670 may represent an assembled internal structure supporting the electronic components of the non-sterile camera 1600, where the electronics scaffold assembled 1670 may facilitate insertion of the internal components into the non-sterile housing body 1660 and retention of the internal components within the non-sterile camera 1600.

In at least some embodiments, FIG. 16 may illustrate an arrangement in which the camera module 1620, the stacked power board, computer, and heatsink 1640, the battery 1650, and the supporting scaffold structures 1630 and 1670 may be integrated into the non-sterile housing body 1660 and retained by the non-sterile housing cap and wire handle 1610 to form the non-sterile camera 1600.

In at least some embodiments, the arrangement shown in FIG. 16 may address thermal management and portable power considerations by including the stacked power board, computer, and heatsink 1640 and the battery 1650 within the non-sterile camera 1600, where such arrangement may support sustained camera operation while permitting later use with a sterile housing or sterile accessory.

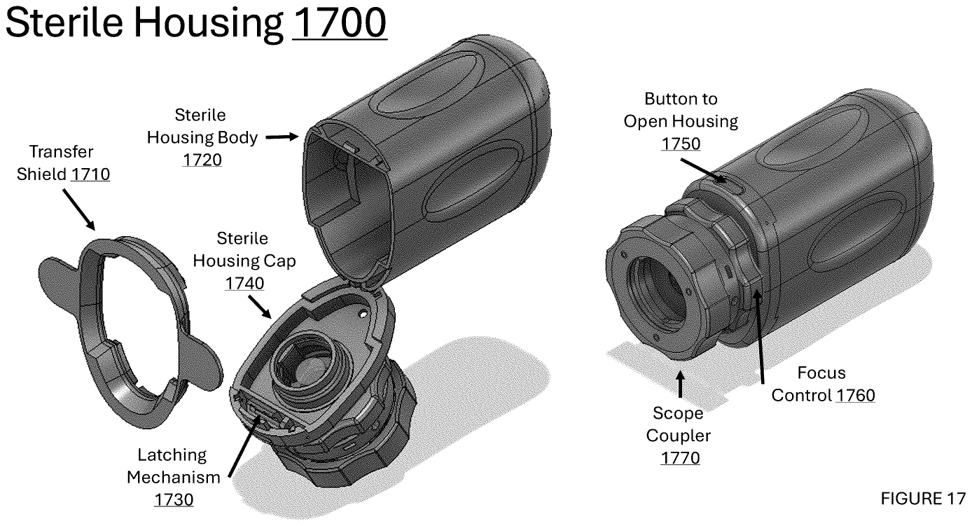

FIG. 17 is an exploded view depicting a sterile housing assembly 1700 in accordance with one or more embodiments of the present disclosure, where the sterile housing assembly 1700 may include a transfer shield 1710, a sterile housing body 1720, a latching mechanism 1730, a sterile housing cap 1740, a button 1750 to open the housing, a focus control 1760, and a scope coupler 1770.

In at least some embodiments, the sterile housing body 1720 may define a primary enclosure portion of the sterile housing assembly, where the sterile housing body 1720 may receive at least a portion of a non-sterile camera assembly and may provide a sterile-contact exterior for clinical use.

In at least some embodiments, the sterile housing cap 1740 may close the sterile housing body 1720 and may cooperate with the sterile housing body 1720 to enclose the non-sterile camera assembly within the sterile housing assembly, where the sterile housing cap 1740 may support assembly and secure retention of the enclosed components.

In at least some embodiments, the latching mechanism 1730 may retain the sterile housing cap 1740 relative to the sterile housing body 1720, where the latching mechanism 1730 may support secure closure of the sterile housing assembly during use in a sterile field.

In at least some embodiments, the button 1750 to open the housing may actuate release of the latching mechanism 1730, where operation of the button 1750 may permit opening of the sterile housing assembly for insertion, removal, and/or replacement of a non-sterile camera assembly.

In at least some embodiments, the focus control 1760 may permit optical focus adjustment while the sterile housing assembly may remain closed, where the focus control 1760 may transmit user input to an underlying camera focus mechanism associated with the enclosed camera assembly.

In at least some embodiments, the sterile housing assembly 1700 may include a lens carried by the sterile housing assembly 1700, and the non-sterile camera assembly may include a camera sensor positioned separately from the lens, where the lens and the camera sensor may be decoupled from one another before assembly and may become optically aligned when the non-sterile camera assembly may be inserted into the sterile housing assembly 1700. In at least some embodiments, insertion of the non-sterile camera assembly into the sterile housing assembly 1700 may align an optical pathway between the lens and the camera sensor, and the focus control 1760 may include a focus knob configured to transform rotational user input into linear travel of at least a portion of a focus structure toward or away from the camera sensor so that image focus may be adjusted while the sterile housing assembly 1700 may remain closed.

In at least some embodiments, the focus control 1760 may include an external knob, ring, lever, or other user-actuatable member that may be mechanically coupled to an internal focus mechanism of an enclosed camera assembly, where movement of the focus control 1760 may be transmitted through the sterile housing assembly 1700 to adjust focus of the camera module while the sterile housing assembly 1700 may remain closed. In at least some embodiments, the mechanical coupling may include a shaft, sleeve, keyed interface, gear interface, friction interface, compliant interface, or other motion-transfer arrangement that may extend through or may engage across a portion of the sterile housing assembly 1700 while maintaining separation between sterile-contact surfaces and non-sterile camera components.

In at least some embodiments, relative rotation, translation, or other actuation of the focus control 1760 may cause corresponding movement of a focus knob, lens mount, threaded focus element, or other camera-side focus structure of the enclosed non-sterile camera assembly, where the focus control 1760 may thereby permit focus adjustment after the non-sterile camera assembly may be loaded into the sterile housing assembly 1700 and attached to an endoscope. In at least some embodiments, the focus control 1760 and the underlying camera-side focus structure may include mating features selected to couple motion while permitting assembly and removal of the non-sterile camera assembly from the sterile housing assembly 1700.

In at least some embodiments, the scope coupler 1770 may mechanically interface with an endoscope, where the scope coupler 1770 may align the optical path of the endoscope with an enclosed camera module and may support attachment of the sterile housing assembly to the endoscope.

In at least some embodiments, the transfer shield 1710 may facilitate transfer of a non-sterile camera assembly into the sterile housing body 1720 while reducing contamination risk, where the transfer shield 1710 may provide an intermediate handling structure during assembly of the sterile housing assembly.

In at least some embodiments, the transfer shield 1710 may include a reusable component of the sterile housing assembly 1700 and may act as a funnel to guide the non-sterile camera assembly into the sterile housing body 1720, where a sterile person may hold the sterile housing body 1720 in an open condition and may place the transfer shield 1710 over an opening of the sterile housing body 1720, and where one or more alignment features between the transfer shield 1710 and the sterile housing body 1720 may position the transfer shield 1710 stably relative to the sterile housing body 1720 during insertion. In at least some embodiments, the transfer shield 1710 may act as a physical barrier that may prevent the non-sterile camera assembly from contacting sterile external surfaces of the sterile housing body 1720 during insertion, where a non-sterile person may insert the non-sterile camera assembly through a central opening of the transfer shield 1710 and into the sterile housing body 1720, and where contact between the non-sterile camera assembly and inward-facing sidewalls of the transfer shield 1710 may be acceptable such that the inward-facing sidewalls may be treated as non-sterile after such contact. In at least some embodiments, after the non-sterile camera assembly may be fully inserted into the sterile housing body 1720, the non-sterile person may remove the transfer shield 1710 while avoiding contact with external-facing surfaces of the sterile housing body 1720, and the sterile person may then close the sterile housing cap 1740 so that non-sterile surfaces of the assembly may be enclosed within the sterile housing assembly 1700.

In at least some embodiments, the transfer shield 1710 may include a barrier structure positioned between the non-sterile camera assembly and sterile-contact portions of the sterile housing body 1720 during insertion, where the transfer shield 1710 may define an opening, channel, funnel, lip, flange, or guide surface that may direct the non-sterile camera assembly into an intended position within the sterile housing body 1720 while reducing contact between the non-sterile camera assembly and exterior sterile-contact surfaces. In at least some embodiments, the transfer shield 1710 may temporarily cover or border at least a portion of the open sterile housing body 1720 during loading and may thereafter be removed before closure of the sterile housing cap 1740.

In at least some embodiments, FIG. 17 may illustrate an arrangement in which the sterile housing body 1720, the sterile housing cap 1740, the latching mechanism 1730, the button 1750, the focus control 1760, and the scope coupler 1770 may cooperate to form a sterile enclosure around a non-sterile camera assembly, while the transfer shield 1710 may facilitate sterile loading of the non-sterile camera assembly into the sterile housing assembly.

In at least some embodiments, the arrangement shown in FIG. 17 may permit a non-sterile camera assembly having active electronics to be used in a sterile environment by isolating the non-sterile components within the sterile housing assembly while still allowing attachment to an endoscope and adjustment of optical focus during a scope-based procedure.

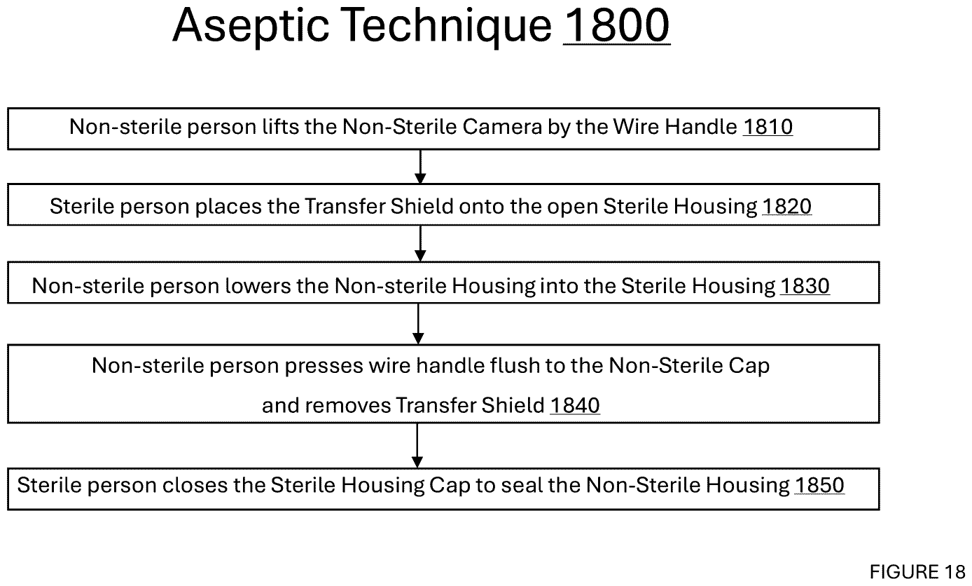

FIG. 18 is a process flow 1800 depicting an aseptic handling technique in accordance with one or more embodiments of the present disclosure, where the process flow 1800 may include a step 1810 in which a non-sterile person may lift a non-sterile camera by a wire handle, a step 1820 in which a sterile person may place a transfer shield onto an open sterile housing, a step 1830 in which the non-sterile person may lower the non-sterile housing into the sterile housing, a step 1840 in which the non-sterile person may press the wire handle flush to a non-sterile cap and may remove the transfer shield, and a step 1850 in which the sterile person may close a sterile housing cap to seal the non-sterile housing.

In at least some embodiments, the step 1810 may include the non-sterile person lifting the non-sterile camera by the wire handle, where the wire handle may facilitate manipulation of the non-sterile camera without direct contact between the sterile person and the non-sterile camera.

In at least some embodiments, the step 1820 may include the sterile person placing the transfer shield onto the open sterile housing, where the transfer shield may provide an intermediate barrier and may facilitate transfer of the non-sterile camera into the sterile housing while reducing contamination risk.

In at least some embodiments, the step 1830 may include the non-sterile person lowering the non-sterile housing into the sterile housing, where the transfer shield and the open sterile housing may cooperate to receive the non-sterile housing while maintaining separation between sterile-contact surfaces and non-sterile handling.

In at least some embodiments, the step 1840 may include the non-sterile person pressing the wire handle flush to the non-sterile cap and removing the transfer shield, where pressing the wire handle flush may position the non-sterile camera for enclosure within the sterile housing and where removal of the transfer shield may prepare the assembly for closure.

In at least some embodiments, the wire handle may include a metal wire bent into a handle shape and may be flexed for insertion into one or more receiving features of the non-sterile housing cap, where the wire handle may act as a spring member and may rotate about one or more insertion locations at the non-sterile housing cap. In at least some embodiments, friction between the wire handle and the one or more receiving features may permit the wire handle to hold a selected rotational position when the wire handle may not be manually manipulated, and pressing the wire handle flush after insertion of the non-sterile camera assembly into the sterile housing assembly may reduce interference with closing of the sterile housing cap.

In at least some embodiments, the step 1850 may include the sterile person closing the sterile housing cap to seal the non-sterile housing, where closure of the sterile housing cap may enclose the non-sterile camera within the sterile housing and may present a sterile-contact exterior for clinical use.

In at least some embodiments, FIG. 18 may illustrate a coordinated transfer process involving both a non-sterile person and a sterile person, where the non-sterile person may handle the non-sterile camera and the sterile person may handle the sterile housing so that active electronic components may be introduced into a sterile-use enclosure in a controlled manner.

In at least some embodiments, the aseptic handling technique shown in FIG. 18 may support use of a non-sterile camera assembly in a sterile field by isolating the non-sterile camera within the sealed sterile housing while preserving optical and mechanical functionality for attachment to an endoscope during a scope-based procedure.

In at least some embodiments, the receiver module may include a non-transitory memory that may store computer code and at least one processor that may execute the computer code to cause the receiver module to receive endoscopic video data from the video capture source over the network connection in accordance with the streaming protocol, to process the endoscopic video data, to generate output data for presentation on a head-mounted display, and to transmit the output data over a wired connection to the head-mounted display.

In at least some embodiments, processing of the endoscopic video data at the receiver module may include decoding the endoscopic video data when the endoscopic video data may be received in a compressed format, where the compressed format may include H.264, and where the receiver module may be configured to support compressed transmission or uncompressed transmission based on the selected streaming protocol.

In at least some embodiments, the video capture source may include a dedicated hardware encoder configured to encode endoscopic video data before transmission to the receiver module, where the dedicated hardware encoder may include a hardware JPEG encoder configured to generate Motion JPEG (MJPEG) output as an alternative to H.264 or another compression format. In at least some embodiments, the MJPEG output may include intraframe-only compression such that individual image frames may be encoded independently without temporal buffering between frames, where the intraframe-only compression may support lower and more deterministic latency than interframe codecs and may provide image quality and latency performance selected for live endoscopic visualization.

In at least some embodiments, the wired connection between the receiver module and the head-mounted display may include a cable connection that may provide low-latency delivery of video to the head-mounted display, where the cable connection may include a Universal Serial Bus Type-C (USB-C) connection, and where the receiver module may be physically separated from the video capture source while the head-mounted display may be worn by the user.

In at least some embodiments, the receiver module may include a smartphone that may execute an application configured to receive endoscopic video data from the video capture source and to output video or output data to XR glasses through a wired interface, where the smartphone may be configured to operate as the receiver module depicted in FIG. 3.

In at least some embodiments, the receiver module may include a Pocket IXR device that may be wearable or portable and may include dedicated receiver-side hardware that may be optimized for onboard encoding, decoding, edge computing, and/or artificial intelligence processing, where the Pocket IXR device may be configured to operate as the receiver module depicted in FIG. 4 and may output video or output data to XR glasses through a wired connection.

In at least some embodiments, the receiver module may include an IXR smartphone dock or server that may be positioned off a sterile field and may expand processing capability for edge computing and/or artificial intelligence processing, where the IXR smartphone dock or server may receive endoscopic video data from the video capture source and may transmit output data to a separate low-profile receiver that may plug into the head-mounted display, as depicted in FIG. 5.

In at least some embodiments, the receiver module may include an IXR box or server that may be positioned off a sterile field and may provide at least one of a wireless access point function, a local area network function, receiver functionality, edge computing capability, or transmission functionality, where the IXR box or server may receive endoscopic video data and may output video or output data for presentation on the head-mounted display, as depicted in FIG. 6.

In at least some embodiments, at least a portion of receiver-side processing capability may be distributed between the video capture source and the receiver module, where the video capture source may perform at least one of encoding, decoding, edge computing, or artificial intelligence processing and the receiver module may perform complementary processing and output to the head-mounted display, as depicted in FIG. 7.

In at least some embodiments, the endoscopic visualization platform may be interoperable with legacy laparoscopic tower equipment, where a transmission module may interface with a tower camera and may transmit endoscopic video data to a smartphone receiver module for output to the head-mounted display, as depicted in FIG. 8.

In at least some embodiments, the endoscopic visualization platform may be interoperable with legacy laparoscopic tower equipment, where a transmission module may interface with a tower camera and may transmit endoscopic video data to a Pocket IXR receiver module for output to the head-mounted display, as depicted in FIG. 9.

In at least some embodiments, a wired connection between a camera module and an IXR box or server may be used to reduce latency and may provide power and compute resources for receiver-side processing, where the IXR box or server may transmit output data to a smartphone receiver module for output to XR glasses, as depicted in FIG. 10.

In at least some embodiments, a wired connection between a camera module and an IXR box or server may be used to reduce latency and may provide power and compute resources for receiver-side processing, where the IXR box or server may transmit output data to a Pocket IXR receiver module for output to XR glasses, as depicted in FIG. 11.

In at least some embodiments, the video capture source may include a coupling mechanism that may lock to an endoscope and may support stable attachment during a procedure, where a focus control may be provided to adjust imaging characteristics while the video capture source may remain attached to the endoscope, as generally depicted in FIG. 12.

In at least some embodiments, the light source may include collet features that may grip a light port of an endoscope and a cap that may engage the collet features to retain the light source on the endoscope, where a switch may control power to one or more light emitters, as generally depicted in FIG. 13.

In at least some embodiments, the receiver module may include a portable housing that may include a battery, a single board computer, and input/output ports that may include a wired output port for connection to the head-mounted display, as generally depicted in FIG. 14.

In at least some embodiments, a housing architecture may separate non-sterile components from sterile-contact surfaces by providing a non-sterile housing that may contain electronics and a sterile housing accessory that may receive the non-sterile housing, as generally depicted in FIGS. 15A and 15B.

In at least some embodiments, thermal management and sterilization constraints may be addressed by providing a non-sterile housing and an internal scaffold that may support a computing module, a power module, and a heat sink arrangement, as generally depicted in FIG. 16.

In at least some embodiments, a sterile housing may include a latching mechanism and an endoscope coupling interface that may allow the assembly to be closed and sealed for clinical use, as generally depicted in FIG. 17.

In at least some embodiments, an aseptic handling technique may include transferring a non-sterile camera housing into a sterile housing using a transfer shield, where a non-sterile person may handle the non-sterile camera housing and a sterile person may handle the sterile housing to reduce contamination risk, as generally depicted in FIG. 18.

In at least some embodiments, the head-mounted display may include XR glasses that may be transparent to allow the user to maintain awareness of the surrounding environment, and the head-mounted display may be configurable to present an endoscopic video window that may be anchored in a field of view or toggled between display modes while endoscopic video may be presented within the user's line of sight.

In at least some embodiments, the endoscopic video data received at the receiver module may be generated by a wireless streaming camera and may be transmitted using a low-latency streaming protocol, where the receiver module may decode the endoscopic video data when the endoscopic video data may be received in a compressed format and may output video to the head-mounted display via a wired connection.

In at least some embodiments, the receiver module may be configured to support optional onboard edge computing and/or artificial intelligence processing, where the optional processing may include object detection, and where a voice interface may be used to control at least one aspect of a visualization workflow using natural language processing.

In at least some embodiments, the receiver module may be configured to output endoscopic video and optional processed data that may support remote consultation, surgical workflow support, and/or generation of clinical documentation, where such processing may be performed locally at the receiver module, at an off-body server module, and/or at a cooperating device based on available compute resources and networking constraints.

In at least some embodiments, the receiver module may include a smartphone running an application that may wirelessly receive endoscopic video data from the video capture source and may wirelessly transmit video or output data to XR glasses, where the smartphone and the XR glasses may communicate over a wireless link instead of a wired connection.

In at least some embodiments, the video capture source may transmit endoscopic video data directly to XR glasses without an intervening smartphone, where the XR glasses may include receiver-side circuitry and/or processing circuitry configured to receive the endoscopic video data and to present the endoscopic video data within a line of sight of a user.

In at least some embodiments, the receiver module may output video or output data from a smartphone to a monitor instead of XR glasses, where the connection between the smartphone and the monitor may be wired or wireless.

In at least some embodiments, the receiver module may include a tablet, a data hub, a server, and/or XR glasses, where the selected receiver module implementation may receive endoscopic video data over a network connection in accordance with a streaming protocol and may output video or output data to a selected user interface.

In at least some embodiments, the user interface may include a head-mounted display, a mobile application, a monitor, and/or a browser, where the endoscopic video data and/or output data may be presented on one or more of such user interfaces individually or in combination.

In at least some embodiments, the light source may include a legacy laparoscopic tower light, where the legacy laparoscopic tower light may be used in place of a custom light source while other portions of the visualization platform may remain unchanged.

In at least some embodiments, the video capture source may include a wireless camera with a sterilizable cover, where the sterilizable cover may at least partially isolate the wireless camera from a sterile field during a procedure.

In at least some embodiments, the light source may include one or more light emitting diodes powered by a battery shared with the video capture source, where the battery of the video capture source may provide electrical power to both image capture components and illumination components.

In at least some embodiments, the video capture source may include a housing having a slimmer and/or more cylindrical profile that may improve ergonomic gripping, handling, and attachment during use with an endoscope.

In at least some embodiments, the video capture source may include a focus mechanism that may provide manual adjustable focus, automatic adjustable focus, or both manual adjustable focus and automatic adjustable focus, where the focus mechanism may facilitate adjustment of image quality while the video capture source may remain attached to the endoscope.

In at least some embodiments, the light source may be provided in different sizes based on a desired battery life, illumination level, procedure duration, and/or ergonomic preference.

In at least some embodiments, the XR glasses may include rims configured to provide increased peripheral visibility during use, where the increased peripheral visibility may facilitate awareness of the surrounding operating room environment while endoscopic video may be presented within the user's line of sight.

In at least some embodiments, the video capture source may include a wireless charging interface, where the wireless charging interface may permit charging of a battery of the video capture source without a physical charging cable.

In at least some embodiments, Wi-Fi access point functionality, transmitter functionality, battery functionality, compute functionality, and receiver functionality may be reduced in size and distributed between a camera/light assembly and a small wireless receiver that may plug directly into XR glasses through a USB-C connection, where the camera/light assembly and the small wireless receiver together may provide a minimized visualization platform.

In at least some embodiments, the application may provide surgical pre-planning support, live remote consultation support during surgery, postoperative consultation support, and/or generation of postoperative notes based at least in part on surgical video and associated patient data.

In at least some embodiments, the disclosed visualization platform may be used in laparoscopy and may additionally be used in other scope-based procedures employing live video feeds, including colonoscopy, hysteroscopy, laryngoscopy, esophagogastroduodenoscopy, thoracoscopy, cystoscopy, and/or arthroscopy.

In at least some embodiments, the system may facilitate real-time consultation with a third-party expert during a procedure, where video from the procedure and/or related output data may be transmitted to support remote guidance and collaboration.

In at least some embodiments, at least one processor of a receiver module that may be separate from a video capture source releasably coupled to an endoscope may receive endoscopic video data transmitted from the video capture source over a network connection in accordance with a streaming protocol, may process the endoscopic video data by decoding the endoscopic video data when the endoscopic video data may be received in a compressed format, may generate output data for presentation on a head-mounted display based on the endoscopic video data, may transmit the output data over a wired connection to the head-mounted display, and may cause presentation of the output data by the head-mounted display within a line of sight of a user during a scope-based procedure.

In at least some embodiments, the streaming protocol may include at least one of a low latency streaming protocol, Real-Time Streaming Protocol (RTSP), or User Datagram Protocol (UDP), where selection of the streaming protocol may depend on a desired latency, compression format, network bandwidth, and/or compatibility with the receiver module and the video capture source.

In at least some embodiments, processing of the endoscopic video data may further include performing, by the at least one processor, at least one of encoding, decoding, edge computing, or artificial intelligence processing on the endoscopic video data, where such processing may be performed locally at the receiver module and/or in cooperation with another device in communication with the receiver module.

In at least some embodiments, the receiver module may include a smartphone running an application configured to receive the endoscopic video data and to output the output data to the head-mounted display over a Universal Serial Bus Type-C (USB-C) connection, where the smartphone may function as the receiver module while the video capture source may remain separate from the smartphone.

In at least some embodiments, the receiver module may include a Pocket IXR device configured to receive the endoscopic video data and to output the output data over a wired connection to XR glasses, where the Pocket IXR device may be portable and may be positioned on the user during the scope-based procedure.

In at least some embodiments, the light source may include a connector interface sized and configured for attachment to a standard endoscope light port, where the connector interface may include light guide connector threads in accordance with ISO/TS 18339:2015, including an M 8.5×0.5 millimeter thread arrangement, and where the light source may include one or more light emitting diodes configured to provide illumination having brightness comparable to a legacy laparoscopic tower light.

In at least some embodiments, the light source may include a portable power source selected to support a desired duration of use, where the desired duration of use may exceed about four hours, and where the light source may be configured for reduced heat emission during operation. In at least some embodiments, the light source may be disposable, reusable, or partially disposable and partially reusable.

In at least some embodiments, the video capture source may include a coupler sized and configured for attachment to a standard endoscope eyepiece, where the coupler may conform to an endoscope eyepiece diameter specification in accordance with ISO/TS 18339:2015, including about 31.75 millimeters plus or minus about 0.1 millimeter. In at least some embodiments, the video capture source may include an ergonomic housing having a slim profile, a cylindrical profile, or a slim and cylindrical profile to facilitate gripping, handling, and attachment during use.

In at least some embodiments, the video capture source may include a sterilizable cover and/or a sterile barrier accessory, where the sterilizable cover and/or the sterile barrier accessory may at least partially isolate the video capture source from a sterile field during a procedure. In at least some embodiments, the video capture source may include a portable power source selected to support a desired duration of use exceeding about four hours and may be configured for reduced heat emission during operation.

In at least some embodiments, the network connection may include a wired communication path, a wireless communication path, or both a wired communication path and a wireless communication path, where the wired communication path may include Ethernet and where the wireless communication path may include Wi-Fi, including Wi-Fi 5, Wi-Fi 6, Wi-Fi 6E, and/or Wi-Fi 7.

In at least some embodiments, the receiver module and/or the UI/UX module may include a power source selected to support a desired duration of use exceeding about four hours and may be configured for reduced heat emission during operation. In at least some embodiments, the UI/UX module may include a display configured to present video at a resolution of about 1080p or higher and may further include an audio interface for user interaction.

In at least some embodiments, a transmission module configured for interoperability with a legacy laparoscopic tower may plug directly into a legacy camera box, where the legacy camera box may be manufactured by any suitable supplier of laparoscopic tower equipment. In at least some embodiments, the transmission module may generate a wireless communication signal and may transmit endoscopic video data from the legacy tower environment to a smartphone receiver module, a Pocket IXR receiver module, and/or another receiver module for downstream presentation.

In at least some embodiments, the application executed by a smartphone, receiver module, server, and/or cooperating device may be trained using videos of laparoscopic surgeries and may provide voice-activated clinical support across multiple phases of care. In at least some embodiments, the application may support surgical pre-planning, intraoperative guidance, remote consultation during surgery, postoperative consultation, generation of postoperative notes, surgical video storage, surgical video editing, surgical video analysis, predictive modeling, and/or computer vision processing.

In at least some embodiments, the disclosed platform may include a context-aware digital assistant configured to support a surgeon across multiple procedures and workflow stages, where the context-aware digital assistant may learn from prior surgical videos, prior interactions, and surgeon-specific practice patterns. In at least some embodiments, the context-aware digital assistant may integrate with existing operating room technologies and future operating room technologies to provide hands-free voice control, contextual recommendations, and workflow optimization.

In at least some embodiments, the disclosed platform may interface with devices and data sources across an operating room and/or a healthcare facility so that procedure-related audio, video, and other operational data may be aggregated on a common platform. In at least some embodiments, such aggregated data may be analyzed using artificial intelligence functionality to generate metrics, workflow insights, utilization information, and/or other output data of interest to clinicians, administrators, and/or healthcare systems.

In at least some embodiments, the video capture source may include a coupler and/or connector sized and configured for attachment to an endoscope, where the coupler and/or connector may be customizable for different endoscope configurations and may be formed using additive manufacturing, including three-dimensional printing, to support selected dimensional, geometric, and fit characteristics.

In at least some embodiments, the video capture source may include a locking mechanism having one or more fins, tabs, or protruding features that may move responsive to relative twisting between portions of the coupler and/or housing, where the one or more fins, tabs, or protruding features may engage the endoscope to secure the video capture source to the endoscope during use and may be released by reverse twisting and/or actuation of a release feature.

In at least some embodiments, a method may include obtaining, by a processing device, endoscopic video data associated with an endoscope, where the endoscopic video data may be received from a video capture source over a network connection in accordance with a streaming protocol and/or may be generated at least in part by the video capture source. In at least some embodiments, the method may further include processing, by the processing device, the endoscopic video data, including decoding the endoscopic video data when the endoscopic video data may be received in a compressed format, generating output data based on the endoscopic video data, transmitting the output data over a wired connection, a wireless connection, or a combination thereof, and causing presentation of the output data at a user interface during a scope-based procedure, where the processing device may be included in the video capture source, may be included in a receiver module, or may be distributed between the video capture source and the receiver module, and where the user interface may include a head-mounted display, XR glasses, a monitor, a mobile application, a browser, and/or another output interface.

In at least some embodiments, a system may include a video capture source configured to obtain endoscopic video data associated with an endoscope, at least one processing device configured to receive and process the endoscopic video data in accordance with a streaming protocol, and a user interface configured to present video or output data during a scope-based procedure, where the at least one processing device may be included in the video capture source, may be included in a receiver module, or may be distributed between the video capture source and the receiver module, and where the user interface may include a head-mounted display, XR glasses, a monitor, a mobile application, a browser, and/or another output interface communicatively coupled through a wired connection, a wireless connection, or a combination thereof.

In at least some embodiments, a method may include receiving, by at least one processor of a receiver module that may be separate from a video capture source releasably coupled to an endoscope, endoscopic video data transmitted from the video capture source over a network connection in accordance with a streaming protocol, processing, by the at least one processor, the endoscopic video data by decoding the endoscopic video data when the endoscopic video data may be received in a compressed format, generating, by the at least one processor and based on the endoscopic video data, output data for presentation on a head-mounted display, transmitting, by the at least one processor, the output data over a wired connection to the head-mounted display, and causing, by the at least one processor, presentation of the output data by the head-mounted display within a line of sight of a user during a scope-based procedure.

In at least some embodiments, a receiver module may include a non-transitory memory storing computer code and at least one processor in communication with the non-transitory memory, where the at least one processor may be configured to execute the computer code to cause the receiver module to receive endoscopic video data from a video capture source releasably couplable to an endoscope over a network connection in accordance with a streaming protocol, to process the endoscopic video data by decoding the endoscopic video data when the endoscopic video data may be received in a compressed format, to generate output data for presentation on a head-mounted display based on the endoscopic video data, and to transmit the output data over a wired connection to the head-mounted display for presentation within a line of sight of a user during a scope-based procedure, where the receiver module may be separate from the video capture source.

In at least some embodiments, the receiver module may include a smartphone configured to receive the endoscopic video data through an application executed by the smartphone, where the application may manage network reception of the endoscopic video data and may provide output of the output data to a head-mounted display.

In at least some embodiments, the receiver module may include a Pocket IXR device including dedicated hardware optimized for at least one of encoding, decoding, edge computing, or artificial intelligence processing, where the dedicated hardware may support low-latency handling of the endoscopic video data for presentation on a head-mounted display.

In at least some embodiments, the receiver module may include an IXR smartphone dock configured to expand processing capability for edge computing or artificial intelligence processing beyond a pocket-type receiver device, where the IXR smartphone dock may interface with a smartphone and may provide additional compute, power, and/or connectivity resources.

In at least some embodiments, the IXR smartphone dock may be configured to transmit the output data to a separate receiver that may plug into the head-mounted display, where the separate receiver may provide a compact interface between the IXR smartphone dock and the head-mounted display.

In at least some embodiments, the receiver module may include an IXR box configured to provide at least one of wireless access point functionality, local area network functionality, receiver functionality, edge computing, or transmission of the output data, where the IXR box may be positioned off a sterile field and may support communication between the video capture source and the head-mounted display.

In at least some embodiments, the receiver module may be configured to cooperate with the video capture source such that processing associated with at least one of encoding, decoding, edge computing, or artificial intelligence processing may be split between the video capture source and the receiver module, where the video capture source may perform a first portion of the processing and the receiver module may perform a second portion of the processing before output to the head-mounted display.

In at least some embodiments, a system may include a video capture source releasably couplable to an endoscope, a receiver module separate from the video capture source, and a head-mounted display communicatively coupled to the receiver module, where the receiver module may include a non-transitory memory storing computer code and at least one processor in communication with the non-transitory memory, and where the at least one processor may be configured to execute the computer code to cause the receiver module to receive endoscopic video data from the video capture source over a network connection in accordance with a streaming protocol, to process the endoscopic video data by decoding the endoscopic video data when the endoscopic video data may be received in a compressed format, to generate output data for presentation on the head-mounted display based on the endoscopic video data, and to transmit the output data over a wired connection to the head-mounted display, where the head-mounted display may be configured to present the output data within a line of sight of a user during a scope-based procedure.

In at least some embodiments, the video capture source may be physically couplable to the endoscope and may be configured to wirelessly transmit the endoscopic video data, where the video capture source may include a camera module and a transmission module associated with the endoscope.

In at least some embodiments, the system may further include a light source configured to physically attach to the endoscope and to illuminate a body cavity being operated in, where the light source may include one or more light emitting diodes and may be battery-powered.

In at least some embodiments, the head-mounted display may include XR glasses communicatively coupled to the receiver module through a USB-C connection, where the XR glasses may present the output data within the line of sight of the user while the receiver module may remain physically separate from the video capture source.

In at least some embodiments, the XR glasses may include an external camera, where the external camera may support capture of an external scene and/or support user interaction or contextual awareness during the scope-based procedure.

In at least some embodiments, the video capture source may include a wireless streaming camera configured to physically attach to the endoscope, where the wireless streaming camera may acquire the endoscopic video data and may transmit the endoscopic video data to the receiver module over the network connection.

In at least some embodiments, the video capture source may include a legacy laparoscopic tower camera and a transmission module configured to transmit the endoscopic video data to the receiver module, where the transmission module may enable interoperability between legacy laparoscopic tower equipment and the receiver module.

In at least some embodiments, the receiver module may include at least one of a smartphone, a Pocket IXR device, an IXR smartphone dock, or an IXR box, where different receiver module configurations may be selected based on desired portability, processing capability, network architecture, and/or clinical workflow considerations.

In at least some embodiments, a method may include receiving, by at least one processor of a receiver module that may be separate from a video capture source releasably coupled to an endoscope, endoscopic video data transmitted from the video capture source over a network connection in accordance with a streaming protocol, processing, by the at least one processor, the endoscopic video data by decoding the endoscopic video data when the endoscopic video data may be received in a compressed format, generating, by the at least one processor and based on the endoscopic video data, output data for presentation on a user interface, transmitting, by the at least one processor, the output data over a wired connection, a wireless connection, or both a wired connection and a wireless connection to the user interface, and causing, by the at least one processor, presentation of the output data by the user interface within a line of sight of a user during a scope-based procedure.

In at least some embodiments, the streaming protocol may include at least one of a low latency streaming protocol, a Real-Time Streaming Protocol (RTSP), or a User Datagram Protocol (UDP).

In at least some embodiments, processing of the endoscopic video data may further include performing, by the at least one processor, at least one of encoding, decoding, edge computing, or artificial intelligence processing on the endoscopic video data.

In at least some embodiments, the receiver module may include a smartphone running an application configured to receive the endoscopic video data and to output the output data to the user interface over a Universal Serial Bus Type-C (USB-C) connection.

In at least some embodiments, the user interface may include a head-mounted display, and the receiver module may include a Pocket IXR device configured to receive the endoscopic video data and to output the output data over a wired connection to the head-mounted display.

In at least some embodiments, a receiver module may include a non-transitory memory storing computer code and at least one processor in communication with the non-transitory memory, where the at least one processor may be configured to execute the computer code to cause the receiver module to receive endoscopic video data from a video capture source releasably couplable to an endoscope over a network connection in accordance with a streaming protocol, to process the endoscopic video data by decoding the endoscopic video data when the endoscopic video data may be received in a compressed format, to generate output data for presentation on a user interface based on the endoscopic video data, and to transmit the output data over a wired connection, a wireless connection, or both a wired connection and a wireless connection to the user interface for presentation within a line of sight of a user during a scope-based procedure, where the receiver module may be separate from the video capture source.

In at least some embodiments, the receiver module may include a smartphone configured to receive the endoscopic video data through an application executed by the smartphone.

In at least some embodiments, the receiver module may include a Pocket IXR device including dedicated hardware optimized for at least one of encoding, decoding, edge computing, or artificial intelligence processing.

In at least some embodiments, the receiver module may include an IXR smartphone dock configured to expand processing capability for edge computing or artificial intelligence processing beyond a pocket-type receiver device.

In at least some embodiments, the IXR smartphone dock may be configured to transmit the output data to a separate receiver that may plug into the user interface.

In at least some embodiments, the receiver module may include an IXR box configured to provide at least one of wireless access point functionality, local area network functionality, receiver functionality, edge computing, or transmission of the output data.

In at least some embodiments, the receiver module may be configured to cooperate with the video capture source such that processing associated with at least one of encoding, decoding, edge computing, or artificial intelligence processing may be split between the video capture source and the receiver module.

In at least some embodiments, a system may include a video capture source releasably couplable to an endoscope, a receiver module separate from the video capture source, and a user interface communicatively coupled to the receiver module, where the receiver module may include a non-transitory memory storing computer code and at least one processor in communication with the non-transitory memory, and where the at least one processor may be configured to execute the computer code to cause the receiver module to receive endoscopic video data from the video capture source over a network connection in accordance with a streaming protocol, to process the endoscopic video data by decoding the endoscopic video data when the endoscopic video data may be received in a compressed format, to generate output data for presentation on the user interface based on the endoscopic video data, and to transmit the output data over a wired connection, a wireless connection, or both a wired connection and a wireless connection to the user interface, where the user interface may be configured to present the output data within a line of sight of a user during a scope-based procedure.

In at least some embodiments, the video capture source may be physically couplable to the endoscope and may be configured to wirelessly transmit the endoscopic video data.

In at least some embodiments, the system may further include a light source configured to physically attach to the endoscope and to illuminate a body cavity being operated in.

In at least some embodiments, the user interface may include XR glasses communicatively coupled to the receiver module through a USB-C connection.

In at least some embodiments, the XR glasses may include at least one external-facing camera.

In at least some embodiments, the video capture source may include a wireless streaming camera configured to physically attach to the endoscope.

In at least some embodiments, the video capture source may include a legacy laparoscopic tower camera and a transmission module configured to transmit the endoscopic video data to the receiver module.

In at least some embodiments, the receiver module may include at least one of a smartphone, a Pocket IXR device, an IXR smartphone dock, or an IXR box.

In at least some embodiments, the method may further include operating a light source in a strobed mode by pulsing one or more light emitters in timed relation with exposure of an image sensor of the video capture source such that illumination may be provided during at least a portion of an exposure interval associated with capture of the endoscopic video data.

In at least some embodiments, the video capture source may include a hardware-based intraframe encoder configured to encode the endoscopic video data before transmission over the network connection to the receiver module.

In at least some embodiments, the video capture source may include a non-sterile camera assembly and a sterile housing assembly including an external focus control mechanically coupled to an internal focus mechanism of the non-sterile camera assembly such that actuation of the external focus control may adjust focus while the sterile housing assembly may remain closed.

In at least some embodiments, the video capture source may include a sterile housing body configured to receive a non-sterile camera assembly and a transfer shield removably positionable at an opening of the sterile housing body to guide insertion of the non-sterile camera assembly into the sterile housing body.

In at least some embodiments, the video capture source may include a non-sterile camera assembly including a housing portion and a wire handle movable between a raised position for transfer of the non-sterile camera assembly and a lowered position against the housing portion during insertion into and closure of a sterile housing assembly.

In at least some embodiments, the user interface may include at least one of a head-mounted display, XR glasses, a monitor, a mobile application, a browser, or another output interface.

In at least some embodiments, the video capture source may include a hardware-based video encoder configured to encode the endoscopic video data with low latency for wireless transmission to the receiver module.

In at least some embodiments, the hardware-based video encoder may include a hardware-based intraframe video encoder.

In at least some embodiments, the hardware-based video encoder may be configured to encode the endoscopic video data according to Motion JPEG (MJPEG) or H.264.

In at least some embodiments, the video capture source may include an endoscope coupler rotatable relative to a housing of the video capture source to facilitate use with an endoscope having an angled tip.

In at least some embodiments, the receiver module may be configured to perform image orientation correction based on rotation of the endoscope coupler relative to the housing of the video capture source.

In at least some embodiments, the receiver module may include a voice interface configured to receive spoken user input and natural language processing configured to interpret the spoken user input to control at least one aspect of presentation of the output data at the user interface.

In at least some embodiments, a method of aseptically preparing an endoscopic camera assembly for use in a sterile field may include providing a non-sterile camera assembly including active electronic components and a wire handle, providing a sterile housing assembly including a sterile housing body, a sterile housing cap, and a transfer shield, placing, by a sterile person, the transfer shield over an opening of the sterile housing body, inserting, by a non-sterile person, the non-sterile camera assembly through the transfer shield and into the sterile housing body, pressing, by the non-sterile person, the wire handle toward the non-sterile camera assembly after the non-sterile camera assembly may be inserted into the sterile housing body, removing, by the non-sterile person, the transfer shield from the sterile housing body after the non-sterile camera assembly may be inserted into the sterile housing body, and closing, by the sterile person, the sterile housing cap relative to the sterile housing body to enclose the non-sterile camera assembly within the sterile housing assembly for use in the sterile field.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

1. A method, comprising:
receiving, by at least one processor of a receiver module that is separate from a video capture source releasably coupled to an endoscope, endoscopic video data transmitted from the video capture source over a network connection in accordance with a streaming protocol;
processing, by the at least one processor, the endoscopic video data by decoding the endoscopic video data when the endoscopic video data is received in a compressed format;
generating, by the at least one processor and based on the endoscopic video data, output data for presentation on a user interface;
transmitting, by the at least one processor, the output data over a wired or a wireless connection to the user interface; and
causing, by the at least one processor, presentation of the output data by the user interface within a line of sight of a user during a scope-based procedure.

2. The method of claim 1, wherein the streaming protocol comprises at least one of a low latency streaming protocol, a Real-Time Streaming Protocol (RTSP), or a User Datagram Protocol (UDP).

3. The method of claim 1, wherein processing the endoscopic video data further comprises performing, by the at least one processor, at least one of encoding, decoding, edge computing, or artificial intelligence processing on the endoscopic video data.

4. The method of claim 1, wherein the user interface comprises at least one of a head-mounted display, extended reality (XR) glasses, a monitor, a mobile application, a browser, or another output interface.

5. The method of claim 1, further comprising operating a light source in a strobed mode by pulsing one or more light emitters in timed relation with exposure of an image sensor of the video capture source such that illumination is provided during at least a portion of an exposure interval associated with capture of the endoscopic video data.

6. A receiver module, comprising:
a non-transitory memory storing computer code; and
at least one processor in communication with the non-transitory memory, wherein the at least one processor is configured to execute the computer code to cause the receiver module to:
receive endoscopic video data from a video capture source releasably couplable to an endoscope over a network connection in accordance with a streaming protocol;
process the endoscopic video data by decoding the endoscopic video data when the endoscopic video data is received in a compressed format;
generate output data for presentation on a user interface based on the endoscopic video data; and
transmit the output data over a wired or wireless connection to the user interface for the presentation within a line of sight of a user during a scope-based procedure,
wherein the receiver module is separate from the video capture source.

7. The receiver module of claim 6, wherein the receiver module is configured to cooperate with the video capture source such that processing associated with at least one of encoding, decoding, edge computing, or artificial intelligence processing is split between the video capture source and the receiver module.

8. A system, comprising:
a video capture source releasably couplable to an endoscope;
a receiver module separate from the video capture source, the receiver module including a non-transitory memory storing computer code and at least one processor in communication with the non-transitory memory;
wherein the at least one processor is configured to execute the computer code to cause the receiver module to:
receive endoscopic video data from the video capture source over a network connection in accordance with a streaming protocol;
process the endoscopic video data by decoding the endoscopic video data when the endoscopic video data is received in a compressed format;
generate output data for presentation on a user interface based on the endoscopic video data; and
transmit the output data over a wired or wireless connection to the user interface; and
the user interface communicatively coupled to the receiver module and configured to present the output data within a line of sight of a user during a scope-based procedure.

9. The system of claim 8, further comprising a light source configured to physically attach to the endoscope and to illuminate a body cavity being operated in.

10. The system of claim 8, wherein the video capture source comprises a legacy laparoscopic tower camera and a transmission module configured to transmit the endoscopic video data to the receiver module.

11. The system of claim 8, wherein the receiver module comprises at least one of a smartphone, a Pocket IXR device, an IXR smartphone dock, or an IXR box.

12. The system of claim 8, wherein the video capture source comprises:
a non-sterile camera assembly; and
a sterile housing assembly comprising an external focus control mechanically coupled to an internal focus mechanism of the non-sterile camera assembly such that actuation of the external focus control adjusts focus while the sterile housing assembly is closed.

13. The system of claim 8, wherein the video capture source comprises:
a sterile housing body configured to receive a non-sterile camera assembly; and
a transfer shield removably positionable at an opening of the sterile housing body to guide insertion of the non-sterile camera assembly into the sterile housing body.

14. The system of claim 8, wherein the video capture source comprises:
a non-sterile camera assembly comprising a housing portion and a wire handle movable between a raised position for transfer of the non-sterile camera assembly and a lowered position against the housing portion during insertion into and closure of a sterile housing assembly.

15. The system of claim 8, wherein the video capture source comprises a hardware-based video encoder configured to encode the endoscopic video data with low latency for wireless transmission to the receiver module.

16. The system of claim 15, wherein the hardware-based video encoder comprises a hardware-based intraframe video encoder.

17. The system of claim 15, wherein the hardware-based video encoder is configured to encode the endoscopic video data according to Motion JPEG (MJPEG) or H.264.

18. The system of claim 8, wherein the video capture source comprises an endoscope coupler rotatable relative to a housing of the video capture source to facilitate use with an endoscope having an angled tip.

19. The system of claim 18, wherein the receiver module is configured to perform image orientation correction based on rotation of the endoscope coupler relative to the housing of the video capture source.

20. The system of claim 8, wherein the receiver module comprises a voice interface configured to receive spoken user input and natural language processing configured to interpret the spoken user input to control at least one aspect of the presentation of the output data at the user interface.
